(19) United States
(12) Patent Application Publication
(10) Pub. No.: US 2016/0032380 A1
(43) Pub. Date: Feb. 4, 2016

(54) ELECTRON BEAM NUCLEIC ACID SEQUENCING

(71) Applicant: Cornell University, Ithaca, NY (US)

(72) Inventors: Harold G. Craighead, Ithaca, NY (US); Leon M. Bellan, Ithaca, NY (US)

(21) Appl. No.: 14/810,413

(22) Filed: Jul. 27, 2015

Related U.S. Application Data
(63) Continuation of application No. 12/819,766, filed on Jun. 21, 2010, now abandoned.
Application No. 12/819,766 is a continuation of application No. 12/049,149, filed on Mar. 14, 2008, now abandoned.
(60) Provisional application No. 60/895,415, filed on Mar. 16, 2007.

(51) Int. Cl. C12Q 1/68; H01J 47/00

(52) U.S. Cl. CPC C12Q 1/6874; H01J 47/00

(57) ABSTRACT

The present invention relates to compositions, methods, and uses for obtaining sequence information from nucleic acid molecules.

### CROSS-REFERENCE

This application is a Continuation application of U.S. patent application Ser. No. 12/819,766, filed Jun. 21, 2010, which is a Continuation application of U.S. patent application Ser. No. 12/049,149, filed Mar. 14, 2008, which claims the benefit of U.S. Provisional Application No. 60/895,415, filed Mar. 16, 2007, each of which is entirely incorporated herein by reference.

### STATEMENT AS TO FEDERALLY SPONSORED RESEARCH

This invention was made with the support of the United States government under Contract number ECCS-9876771 by Nanobiotechnology Center of the National Science Foundation (NSF).

### BACKGROUND OF THE INVENTION

Our increased understanding of the specific roles of biomolecules in determining all aspects of biological phenomena has increased the need for rapid and accurate determination of sequence, structure and properties of large numbers of biomolecules. Such need has led to considerable interest in the structure and analysis of single biomolecules. Part of the growing interest is due to the rapid development of methodologies for the manipulation and detection of single macromolecules. For example, recent developments in experimental techniques and available hardware have increased dramatically the sensitivity of detection so that optical detection can be made of single dye molecules in a sample. Single dye detection can be done in an aqueous solution, at room temperature (see, e.g., Weiss, 1999, Science 283: 1676-1683), and in very small volumes to reduce background. Such single-molecule based analytical methods are especially useful in the analysis of biopolymers, such as nucleic acids, proteins, and carbohydrates. Single-molecule analytical methods require small amounts of sample, thereby alleviating tedious efforts in generating large amounts of sample material. For example, single-molecule analytical methods may allow analysis of the structure of nucleic acid molecules without amplification, by e.g., polymerase-chain reaction (PCR). Single-molecule analytical methods also allow analysis of individual molecules, and are thus particularly useful in the identification of structural and/or dynamical features without the effect of averaging the properties being examined over a heterogeneous population. While the techniques for analyzing single molecules have developed rapidly, there is still a need for methods to isolate, store, and manipulate biomolecules in order to determine their sequences, structure and properties.

Approaches to nucleic acid sequencing have varied widely, and have made it possible to sequence entire genomes, including portions of the human genome. The most commonly used method has been the dideoxy chain termination method of Sanger (1977, Proc. Natl. Acad. Sci. USA 74:5463). Automated DNA sequencing systems based on this technology have been developed which used four fluorescently labeled dideoxy nucleotides to label DNA. However, these methods are still dependent on Sanger sequencing reactions and gel electrophoresis to generate ladders and robotic sample handling procedures to deal with the attending numbers of clones and polymerase chain reacting products.

Recent advances in methods of single molecule detection (described, for example, in Trabesinger, W., et al., Anal Chem., 1999. 71(1); p. 279-83 and WO 00/06770) make it possible to apply sequencing strategies to single molecules. Another method is based on base excision and described, for example, in Hawkins, G. and L. Hoffman, Nature Biotechnology, 1997. vol. 15; p. 803-804 and U.S. Pat. No. 5,674,743. With this strategy, single template molecules are generated such that every base is labeled with an appropriate reporter. The template molecules are digested with exonuclease and the excised bases are monitored and identified. There still exists a need for sequencing methods that are efficient, reliable, and can be performed on stored nucleic acids.

Gene expression profiling allows for a determination of the level to which a set of genes is being expressed in a given set of cells at a given time, and provides a powerful means for detecting and understanding normal or aberrant cellular behavior. DNA microarrays are now widely used for expression profiling, because they are intrinsically massively parallel and experimentally accessible. Brown & Botstein, 1999, Nature Genetics 21:33-37. Two main technologies are commonly used to produce DNA chips: photolithography as developed by Affymetrix and mechanical grid systems, which deposit PCR products or clones into two-dimensional arrays. Celis et al., 2000, FEBS Letters 480:2-16. While these approaches analyze the expression levels of thousands of genes simultaneously, they each suffer from significant limitations, such as scalability, speed, and ease of automation.

Significant efforts have been directed to investigating techniques to stretch DNA molecules in order to both better understand the molecular dynamics and develop single molecule genomic analysis techniques. In many of these techniques, one end of the DNA molecules is bound to a surface while the other is manipulated with a controllable force using, for example, magnetic tweezers (see Smith et al., Science, 258, 1122, 1992), optical tweezers (see Smith et al., Science, 271, 795, 1996), or an atomic force microscope (AFM) probe (see Rief et al., Nature Struct. Biol., 6(4), 346, 1999, and Shivashankar et al., Appl. Phy. Lett., 71(25), 3727, 1997). The DNA molecules may also be stretched by immobilizing one end and allowing the molecule to experience a hydrodynamic flow (see Perkins et al., Science, 268, 83, 1995). Other methods for stretching DNA molecules include forcing them into nanochannels (see Mannion et al., Biophys. J., 9 (12), 4538, 2006 and Reccius et al. Phys. Rev. Lett., 95, 2005), molecular combing (see Bensimon et al., Science, 265, 2096, 1994) and causing them to experience elongational flow (see Perkins et al., Science, 276, 2016, 1997 and Smith et al. Science, 281, 1335, 1998). The majority of these techniques do not result in molecules that remain stretched after the experiment or can only stretch a few molecules at a time. None of these techniques produce stretched DNA molecules encapsulated in a protective medium that can be subsequently manipulated and analyzed optically or mechanically.

Thus there remains a considerable need for alternative devices and methods for isolating, storing, and priming biomolecules for further analyses.

### SUMMARY OF THE INVENTION

The present invention provides compositions and methods for fixing individual biomolecules in a fiber, allowing the molecules to be stored, retrieved, detected, and analyzed for sequence, structure, and other properties. One aspect of the present invention utilizes the elongational flow of an electrospinning jet to simultaneously stretch DNA molecules and encapsulate them in a polymeric nanofiber for subsequent investigation and manipulation.

One aspect of the invention is a fiber comprising at least one biomolecule that is fixed therein.

In some embodiments, the biomolecule is a biopolymer. In some embodiments, the biopolymer is elongated. The biopolymer can be selected, for example, from the group consisting of a nucleic acid, polypeptide, lipids, carbohydrate, and a combination thereof. The biopolymer can contain DNA, RNA or amino acids or sugar units (e.g., glucose, fructose, galactose and the like). In some embodiments the biopolymer is associated with a metal. In some embodiments the biopolymer is labeled, for example with a fluorophore. The fiber can also have a plurality of biomolecules, each of which is individually observable.

In some embodiments, the biopolymer comprises individual units, and the units are individually observable. For example, the biopolymer can be nucleic acid and the unit is a nucleotide.

The fiber can have a biomolecule that is observable by one or more mechanisms selected from the group consisting of absorbance, fluorescence, luminescence, or scattering. In some embodiments, the biomolecule is observable via interaction with electron, photon or neutron.

In some embodiments the fiber is a nanofiber. The fiber may have a cross-sectional dimension ranging from about 25 nanometers to about 2 micrometers. In some embodiments, the fiber has a cross-sectional dimension of less than about 150 nanometers. In some embodiments, the fiber material comprises a polymer, for example, a water compatible polymer. In some embodiments, the fiber is cross linked. In some embodiments the fiber is cross-linked after it is deposited onto a substrate.

One aspect of the invention is an array having a substrate deposited thereon a fiber comprising at least one biomolecule that is fixed therein. The array can comprise a plurality of fibers. In some embodiments, the fiber is oriented on the array with or without addressable locations. In other embodiments, the fiber is deposited on a disk.

One aspect of the invention is a system for detecting a biomolecule comprising: a fiber comprising at least one biomolecule that is fixed therein; and a detection system operatively coupled to said fiber, wherein the detection system detects a signal from the biomolecule. In some embodiments, the system is an optical system capable of detecting an optical signal coming from the biomolecule. In some embodiments, the system is an electron microscopy system.

One aspect of the invention is a method of isolating a biomolecule comprising: mixing a biomolecule into a fiber forming material; and forming a fiber that comprises the biomolecule fixed therein, thereby isolating said biomolecule. In some embodiments, the step of forming a fiber comprises electrospinning. In some embodiments, the biomolecule is a biopolymer, and in some embodiments, the biopolymer is elongated. The biopolymer can be selected, for example, from the group consisting of a nucleic acid, polypeptide, carbohydrate, and a combination thereof. In some embodiments, the biomolecule is labeled, for example with a fluorophore. The method can comprise a plurality of biomolecules, each of which being individually observable. In some embodiments, the biopolymer comprises individual units, and the units are individually observable.

In some embodiments, the biomolecule is pre-treated by subjecting it to a chemical reaction, for example, hybridization, enzymatic reaction, and protein/nucleic acid binding.

One aspect of the invention is a method of analyzing a biomolecule comprising: providing a fiber fixed therein an isolated biomolecule that is configured to produce a detectable signal; and detecting said signal thereby analyzing said biomolecule. In some embodiments, the biomolecule is labeled, and in some cases, said detection is effected by photon absorbance, fluorescence, luminescence, or scattering.

One aspect of the invention is a method of sequencing a target nucleic acid molecule comprising: (i) providing a fiber fixed therein the target nucleic acid molecule; (ii) subjecting the target nucleic acid molecule to an endonuclease or exonuclease reaction to yield a sequence of cleaved fragments; and (iii) detecting the sequence of the cleaved fragments. In some embodiments, the target nucleic acid molecule is labeled, and in some embodiments, each nucleotide unit of the target nucleic acid molecule is labeled. In some embodiments, the exonuclease reaction comprises a 3′ to 5′ exonuclease. In some embodiments, the cleaved fragments are individual nucleotides. In some embodiments, the endonuclease reaction comprises a restriction exonuclease.

In some embodiments, the target nucleic acid molecule is a DNA molecule. In some embodiments, the label is attached to the nucleotide unit at its base, sugar moiety, alpha phosphate, beta phosphate, and/or gamma phosphate.

In some embodiments, the target nucleic acid comprises a plurality of types of nucleotides, wherein each type has a different label which is distinguished from one another during said registering step.

One aspect of the invention is a method of detecting the presence of an interaction involving a target biopolymer and a probe comprising: providing a fiber fixed therein an isolated biopolymer; contacting the probe with the biopolymer under conditions sufficient to produce a stable probe-target biopolymer complex; detecting the formation of the stable probe-target complex, thereby detecting the presence of the interaction.

In some embodiments, the biopolymer is a nucleic acid and the probe is a nucleic acid probe or a proteinaceous probe. In some embodiments the biopolymer is a protein and the probe is a nucleic acid probe or a proteinaceous probe.

In some embodiments the interaction involves a target nucleic acid biopolymer and a transcription factor.

In some embodiments the interaction involves a target nucleic acid biopolymer and an endonuclease or an exonuclease. The exonuclease can be, for example, a DNA nuclease, or it can be an RNA nuclease.

In some embodiments, the interaction is detected by light, electrons, or neutrons.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows a micrograph of a fiber deposited in a curved pattern onto a substrate by moving the substrate in the x direction, and simultaneously using the voice coil of a loudspeaker to move the fiber in the y direction.

FIG. 2(a) is a drawing illustrating the deposition of an electrospun fiber onto a rotating substrate.

FIG. 2(b) is a drawing illustrating control of the deposition of a fiber onto a substrate by controlling the x-y movement of the substrate.

FIG. 3 is a schematic representation of electron beam reading of the order and identity of labeled bases in an isolated double-stranded nucleic acid elongated within a fiber.

FIG. 4 (a)-(c) are micrographs of isolated, fluorescently labeled lambda DNA molecules elongated in electrospun fibers (inserts are the same fibers at higher contrast making fiber autofluorescence visible).

FIG. 4(d) is a micrograph of fibers electrospun as in FIGS. 4(a)-(c) without the fluorescent dye.

FIG. 4(e) is a micrograph of fibers electrospun as in FIGS. 4(a)-(c) without the addition of poly (aspartate) to the DNA.

FIG. 4(f) is a micrograph of ribbons deposited onto a substrate containing isolated, elongated DNA molecules.

FIG. 5 is a histogram of the measured lengths of elongated lambda DNA in electrospun fibers.

### DETAILED DESCRIPTION OF THE INVENTION

Described herein are compositions and methods for isolating, manipulating, and analyzing individual biomolecules including biopolymers. The compositions and methods include single nucleic acid molecules, which can be isolated within the fiber, stored, and subsequently analyzed. The biopolymers can be elongated and fixed within the fibers, and the elongated molecules can subsequently analyzed for sequence, structure, and properties.

### DEFINITIONS

The term “electromagnetic radiation” refers to electromagnetic waves of energy including, for example, in an ascending order of frequency (or alternatively, in a descending order of wavelength), infrared radiation, visible light, ultraviolet (UV) light, X-rays, and gamma rays. Electromagnetic radiation as used herein is synonymous with the term “light”. Electromagnetic radiation may be in the form of a direct light source or it may be emitted by a light emissive compound such as a donor fluorophore.

The term “polymer” is used herein to refer both to the fiber material, which in some embodiments is polymer, and to the isolated biomolecule, which, in some embodiments is a biopolymer. While in some cases it may be advantageous to use a biopolymer as all or a portion of the fiber material, the use of the biopolymer is distinct from that of the isolated biopolymer in the fiber. The polymer molecules that make up the fiber material are generally present in amounts such that no single molecule would be isolated within the fiber.

### Fiber of the Present Invention:

The present invention provides compositions for isolating biomolecules. In particular, the present invention provides a fiber comprising at least one biomolecule that is fixed therein. A fiber refers to a structure having a volume for trapping a biomolecule. Contemplated herein are all longitudinal structures, and transverse segments of such longitudinal structures, which can be of variable size, shape, and volume. It is not intended to be limited as regard to the material from which and the manner in which it is made. A fiber has a longitudinal axis substantially parallel with the wall of the fiber, and perpendicular to the longitudinal axis, a plane comprising two cross sectional axes along which transverse segments of a fiber can be sectioned. The longitudinal axis may be the same length, or shorter but usually longer than the horizontal axis. The transverse segments of a fiber may also vary in shape and dimensions. The fiber can be solid, or may contain voids, either being microporous, or having larger openings, for example, tubular in shape.

The fiber provides a means for fixation of the molecule, allowing the orientation and the physical structure of the molecule to be held in place by the material in the fiber. Where the biomolecule is a biopolymer, it can be elongated from its random coil configuration within the fiber, and in some cases elongated such that it is held linearly in the fiber with its chain axis parallel to the fiber longitudinal axis. A fiber is a convenient structure for analysis of the biomolecule fixed within it. The fiber can allow the biomolecule to be viewed 360 degrees around the cross section of the fiber and also at angles with respect to the chain dimension. The fiber can be observed both in reflection mode, and in transmission mode, for instance, but suspending the fiber over an opening. A fiber can be deposited in place or moved and manipulated. While held in place, the fiber can be analyzed, for instance, chemically, optically, or with X-rays, electrons or neutrons. The fiber can be deposited onto a substrate so that the biomolecule can be held in an addressable location which can later be accessed.

It is often desirable that the fiber be thin. A thin fiber allows the biomolecule to be observed with less material around it than would be the case if the fiber were thick. A thin fiber can be desirable in some embodiments because the material surrounding the biomolecule can, in some cases, interfere with the measurement of the property of interest of the biomolecule. For instance, where the fluorescence is being detected, the fiber material can result in scattering, absorption, or fluorescence which can interfere with the detection of the fluorescence related to the biomolecule. The desired thickness of the fiber is in some cases depend on the dimensions of the biomolecule isolated within the fiber, and the dimensions of biomolecules span a wide range, with small biomolecules molecules and enzymes having un-elongated dimensions on the order of 0.1 to 200 nanometers, and incompletely compacted human chromosomes during mitosis (used for Karyotyping) having dimensions on the order of 0.5 to 20 micrometers (500 to 20,000 nanometers). A fully elongated double stranded DNA from a human chromosome would extend to on the order of 17 millimeters to 83 millimeters, but would be only 0.2 nanometers thick. Thin fibers can also allow for the packing of more information into a smaller space. For instance, where the fibers are deposited onto a substrate, if the fibers are thin, and the fibers are deposited next to one another over the area, a longer total length of fiber can be deposited in a given area for a thin fiber. Thin fibers also provide more facile access of reagents to treat the biomolecule, for instance by binding, labeling, or cutting the biomolecule, for instance with restriction enzymes. The rate of penetration of a reagent into a material is dependent on the thickness of the material. The thinner the fiber of a given material, generally the more rapidly a reagent would be able to access the biomolecule. One of skill in the art would appreciate that the thickness of the fibers can be adjusted for a particular application to avoid breakage or other physical damages.

In some embodiments, at least one cross-sectional dimension of the fiber is at least about 10, 100, 1,000, 10,000, or 100,000 nanometers. In some embodiments at least one cross sectional diameter of the fiber is between about 10 and 50 nanometers, between about 10 and about 100 nanometers, between about 50 and 150 nanometers, between about 100 and about 1,000 nanometers, between about 10 and about 1,000 nanometers, between about 100 and about 1,000 nanometers, or between about 100 and about 10,000 nanometers. In some embodiments, a cross sectional dimension of the fiber is about 100 nm.

Because the desired thickness of the fiber depends on the size of the isolated biomolecule, it can be useful to express the thickness in term of the multiple of a dimension of the isolated biomolecule. In some embodiments the thickness of the fiber is less than about 2, 5, 10, 50, 100, 500, 1,000, 5,000, or 10,000 times the greatest cross-sectional dimension of the isolated biomolecule within the fiber.

In embodiments where electrons are used for detecting the isolated biomolecule in the fiber, it is particularly desirable that the fiber be thin. The labels used for detection with electron microscopy are often based on atomic density, and when the labeled material is embedded within a surrounding material, the density fluctuations in that material can mask the information related to the isolated biomolecule. This is particularly desirable where electron microscopy is used and high spacial resolution is required, such as to determine the sequence of a nucleic acid biomolecule. For some embodiments utilizing electrons for detection, at least one cross sectional dimension of the fiber is less than about 500, 200, 150, 100, 50, 10, or 5 nanometers.

The cross-sectional shape of the fiber can be round, ellipsoidal, square, rectangular, oval, or any other shape. The cross-section can also exhibit voids, either due to being porous, or, for example being tubular. The fiber is often a fluid at some point prior to the fiber formation, and as a fluid, surface tension forces tend to favor a round cross section. Other effects such as gravity can cause the shape to deviate from round. As the fiber hardens into a solid, forces such as crystallization can also give rise to a non-spherical cross section. Where the solid fiber is stretched, the surface tension forces may no longer control the cross sectional shape of the fiber which will not be round. Physical distortion can shape the cross section of the fiber. The physical distortion can occur during fiber formation. For instance, where fibers are prepared by extrusion through a dye, the shape of the dye can impart a shape on the fiber. In addition, physical distortion can be applied subsequent to fiber formation. For instance, deposition of the fiber onto a surface may result in a distortion such as flattening of the fiber. In some cases, the fiber can be pressed, for example, run through rollers to control its shape. In some embodiments a round fiber is desirable to effect analysis of the fixed biomolecule at any angle around the cross section of the fiber. In some embodiments, a fiber with a cross-sectional shape that is smaller in one dimension than in another, for example flattened, ellipsoidal, rectangular, elliptical, or ribbon shape, can be beneficial. The aspect ratio of the cross-section can also affect the accessibility of the biomolecule to reagents used to bind, treat, or label the biomolecule within the fiber. The amount of time that it takes for a reagent to penetrate a material is related to the thickness of the material. The isolated biomolecules in a fiber with a higher aspect ratio will therefore be more readily accessible to a reagent than would a biomolecule in a fiber with a low aspect ratio at the same cross-sectional circumference. The fiber's narrow cross sectional dimension can enhance visualization, and the fibers larger cross sectional dimension can provide mechanical integrity. In some embodiments, the ratio of the cross sectional dimensions is about 1:1, 1:2, 1:5, 1:10, 1:50, 1:100 or higher.

In order to detect the biomolecule within the fiber, it is often desirable that the fiber transmit the form of radiation that is used to detect the biomolecule. For instance, where the detection of the biomolecule involves the use of light, the fiber will generally be configured to allow enough light to penetrate into the fiber to interact with the biomolecule and/or to penetrate out of the fiber to effect detection. Where desired, the fiber material is non-opaque to the form of radiation used to detect the biomolecule. Materials that are substantially transparent (i.e., permitting the majority of the radiation passes through the material without being absorbed, reflected, scattered, or otherwise altered) are generally preferred. However, the fiber material need not be completely transparent to the radiation used to detect the biomolecule, but must be transparent enough to allow enough of the radiation in, and/or out of the fiber to detect the biomolecule in the intended application. In the case of light, for most materials, the transparency of the material will depend on the wavelength of the light. Thus a material may, for example, be highly transparent at visible wavelengths and highly opaque in the ultraviolet (UV) portion of the spectrum, and thus would be useful as a fiber to detect a biomolecule with optical wavelength light, but not with UV light. The amount of light absorption of a material as a function of the wavelength of light, is sometimes referred to as the absorbance spectrum of the material. One of ordinary skill in the art would appreciate the use of the absorption spectrum in order to identify fiber materials with sufficient transparency for the wavelength range of interest.

Fibers embedded with biomolecules can be made of a variety of materials including polymers, silicones, glasses, and sol-gel ceramics. The type of material that can be used will depend on the type of process which is employed to make the fiber.

### Fabrication of the Subject Fibers:

A variety of methods are available for fabricating fibers useful for isolating biomolecules. Non-limiting examples include melt spinning, polymerization from monomeric liquids, and solvent spinning Melt spinning involves melting the polymer or glass by raising the temperature to melt the material to form a liquid, or molten state. The molten state is the precursor to fiber formation. The molten material is then formed into a thin stream, which then cools and solidifies into a fiber. The cooled, solidified fiber can be further handled and processed. A conventional melt spinning technique uses an extruder which melts and mixes the material. The extruder is equipped with a die which can have one, or a plurality of openings, or dies, through which the molten material is passed. By controlling factors such as the time, temperature, viscosity, and shear rate, the strain rate within the melt can be controlled, allowing for elongation of biomolecules as they pass through the die. Solvent spinning can be similarly performed, but the precursor to the fiber is a solution including a low molecular weight precursor which can be removed by volatilization after the fiber exits the die. By controlling the viscosity, rate, temperatures, geometries, shear and strain rates, biomolecules can be elongated during solvent spinning. In some cases, solvent spinning can be performed without a die by pulling a fiber directly from solution.

A second type of fiber forming process uses a monomeric liquid as the precursor to the fiber. The monomeric liquid is formed into a thin stream, and the monomeric liquid is polymerized to form a polymer, which solidifies to form the fiber. A third type of fiber forming process uses a solution of the polymer dissolved in a solvent. The liquid polymer solution is formed into a thin stream, the solvent is rapidly evaporated from the stream and the polymer solidifies into a fiber. Combinations of the three basic methods can also be used. All three of these methods can be used both with conventional polymer processing or with electrospinning to produce fibers with isolated biomolecules.

Materials useful for the melt spinning process must generally be thermoplastic materials that reversibly convert between solid and liquid forms with temperature. Materials useful for melt spinning include both glasses and thermoplastic polymers. The thermoplastic polymers useful for melt spinning are, for example, polyethylene, polypropylene, polybutene, poly-4-methyl pentene, polystyrene, and the like; cyclic olefin polymers, modified polyolefins, such as copolymers of various alpha-olefins, glycidyl esters of unsaturated acids, ionomers, ethylene/vinyl copolymers such as ethylene/vinyl chloride copolymers, ethylene/vinyl acetate copolymers, ethylene/acrylic acid copolymers, ethylene/methacrylic acid copolymers and the like, thermoplastic polyurethanes, polyvinyl chloride, polyvinlidene chloride copolymers, liquid crystalline polymers, fluorinated polymers such as polytetrafluoroethylene, ethylene tetrafluoroethylene copolymers, tetrafluoroethylene hexafluoropropylene copolymers, polyfluoroalkoxy copolymers, polyvinylidene fluoride, polyvinylidene copolymers, ethylene chlorotrifluoroethylene copolymers, and the like, polyamides, such as the nylons, and the like, polyimides, polyphenylene sulfide, polyphenylene oxide, polysulfones, polyethersulfones, polycarbonate, polyacrylates, terpene resins, polyacetal, styrene/acrylonitrile copolymers, styrene/maleic anhydride copolymers, styrene/maleimide copolymers, and the like and combinations thereof. Where melt spun polymers are used to produce the fiber, it is desirable that the time, temperature, and shear in the processing be controlled in order to avoid degradation of the biomolecule. Low temperature glass may be a desirable choice of fiber material for the melt spinning process.

Materials useful for forming fibers from a monomeric liquid require that there be a liquid monomer which can be rapidly polymerized to form the solidified fiber. In general, these systems require a thermoset system. The monomers can be reactive species such as olefins, for example acrylate, methacrylate; a combination of an isocyanate, and a polyamine or polyol resulting in a polyurethane; a silane and a vinyl monomer, curable, for example with a platinum catalyst to form a silicone; or a combination of an epoxide and an amine monomer in order to form an epoxy. These systems can be cured, for example with UV light or with a thermal cure.

Materials useful for the process of forming fibers from a polymer solution in solvent require that the fiber material be dissolved in a solvent that can be rapidly removed to form the fiber. The materials listed above for melt processing can also generally be used in this process when dissolved in the appropriate solvent. This process allows the formation of fibers from an aqueous solution where the fiber polymers are water soluble. Examples of water soluble polymers that are useful in forming fibers with isolated biomolecules are polyethers, such as polyethylene glycol (PEG), polyethylene oxide (PEO), PEO-PPO) block or random copolymers; polyvinyl alcohol (PVA); poly(vinyl pyrrolidinone) (PVP); poly acrylic acid, poly methacrylic acid, poly(amino acids) such as polyaspartate and polyglutamate; dextran; proteins; hydroxypropyl cellulose (HPC), hydroxypropyl methyl cellulose (HPMC), methyl cellulose, poly acrylamide, agarose, and poly (N-vinyl-2-pyrrolidinone) (PVP). Polymers containing functional groups like hydroxyl, amine, sulfonate and carboxylate tend to be water soluble and may be useful as fiber materials. It can be desirable to have a fiber material that exhibits good mechanical properties in order to maintain the integrity of the fiber during handling, deposition, and manipulation. In the case of the water soluble polymers, it is not necessary that the polymer be completely soluble in the solution. The polymer could be, for instance, partly soluble in the solvent as long as it is compatible enough with the solvent to form a fiber as the solvent dissolves. In some cases, combinations of the water soluble polymers will provide a superior combination of properties.

Electrospinning is another method for fabricating the subject fibers. Fiber forming materials amenable to this procedure include but are not limited to glass or ceramics (see, for example, Kameoka, J., Fabrication of suspended silica glass nanofibers from polymeric materials using a scanned electrospinning source, Nano Lett., 4(11), 2105, 2004; and Li et al., Direct fabrication of composite and ceramic hollow nanofibers by electrospinning, Nano Lett., 4(5), 933, 2004). In electrospinning, typically a high voltage is applied to viscous solution on a sharp conducting tip, causing it to form a Taylor cone (see Reneker et al., Nanometre diameter fibres of polymer, produced by electrospinning, Nanotechnology, 7(3), 2161996). As the electric field is increased, a fluid jet is extracted from the Taylor cone and accelerated towards a grounded collecting substrate. In one embodiment of the present invention, in-flight solvent evaporation is used to form solid polymer fibers from a polymer solution. In another embodiment, melt spinning can also be used. Melt electrospinning of fibers is known in the art (see Larrondo et al. Electrostatic fiber spinning from polymer melts, I. J. Polym. Sci., Polym. Phys. Ed., 19(6), 909, 1981). In other embodiments of the present invention, electrospinning with in-flight polymerization or crosslinking can be used to produce elongated isolated biopolymers. These methods are also known in the art (see, for example, Gupta et al., Macromol., 37(24), 9211, 2004; and Kim et al., Macromol., 38(9), 3719, 2005). One advantage of electrospinning is the lack of harsh chemical processes, allowing for the use of electrospinning for biological applications, for instance, biological entities such as viruses and enzymes have been incorporated into fibers (see Lee et al. Virus-Based Fabrication of Micro- and Nanofibers Using Electrospinning Nano Lett., 4(3), 387, 2004; and Patel et al. Nano Lett., 6, 1042, 2006).

Electrospinning is particularly advantageous for producing the elongated biopolymers of the present invention because the process can be run in a manner which creates large strain rates in electrospinning jets, resulting in fibers which can contain highly oriented polymer molecules, (see Reneker et al., J. Appl. Phys., 87(9), 4531, 2000). Electrospinning has also been used to orient anisotropic particles such as carbon nanotubes, and CdS nanowires in fibers. It is known in the art that DNA can be made into fibers using electrospinning (Fang et al., DNA fibers by electrospinning, J. Macromol. Sci. Phy., B36(2), 169, 1997; Takahashi et al. Fabrication of DNA nanofibers on a planar surface by electrospinning, Jap. J. Appl. Phys., 44, (27), L860, 2005). While these references showed that DNA fibers could be successfully produced by electrospinning, they did not demonstrate production or even an attempt to produce isolated DNA molecules. In these papers, fibers were spun from DNA in water-alcohol solution to produce fibers that were made completely of DNA. Since the fibers were composed solely of DNA, the DNA molecules in the fibers were not isolated. DNA has been incorporated into electrospun nanofiber membranes at relatively high concentrations for the purposes of making a controlled release membrane (Luu et al. Development of a nanostructured DNA delivery scaffold via electrospinning of PLGA and PLA-PEG block copolymers, J. Contr. Rel., 89(2), 341, 2003), demonstrating that DNA can remain structurally intact and bioactive after electrospinning.

The subject fibers can be prepared in an array format by electrospinning, other methods disclosed herein or available in the art. The substrate onto which the fiber is deposited can be of any material, for instance a polymer, glass, ceramic, metal or semiconductor. The substrate can be transparent, opaque, conductive or insulating. In some cases, the substrate can have active elements such as detectors or emitters of light, or electricity including charge, current, or voltage.

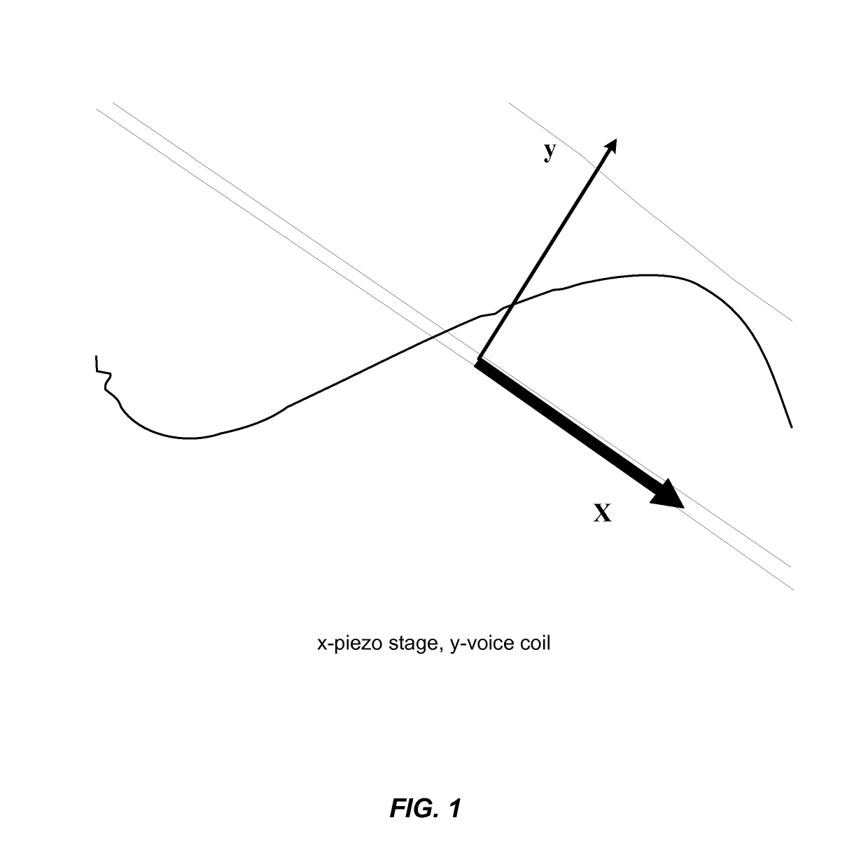

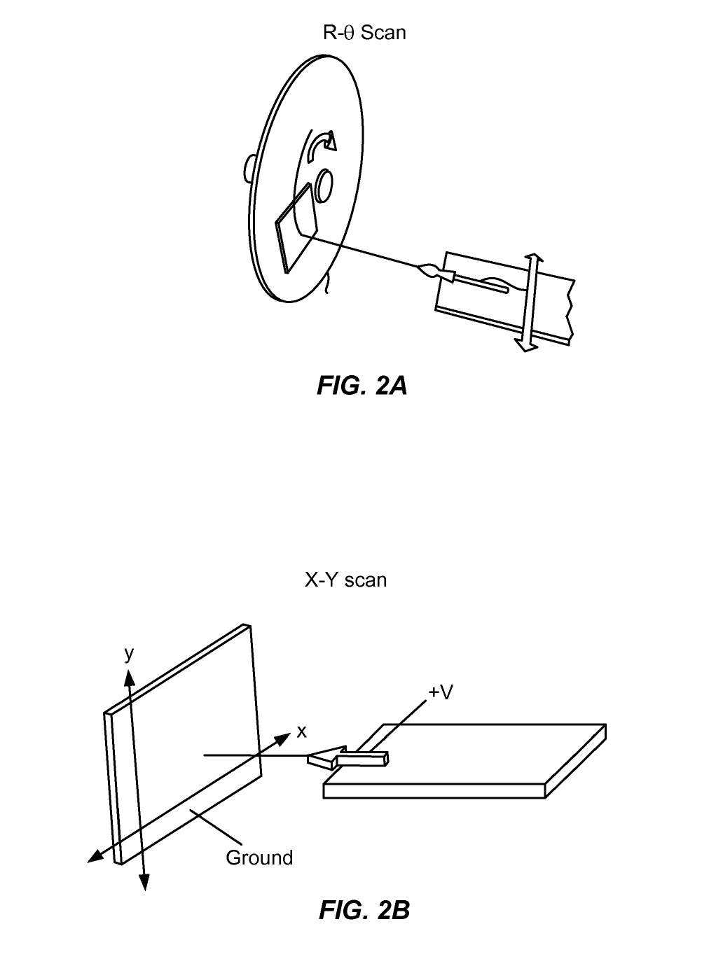

The subject fibers may align horizontally or diagonally long the x-axis or the y-axis of the substrate. The individual fibers can be arrayed in any format across or over the surface of the substrate, such as in rows and columns so as to form a grid, or to form a circular, elliptical, oval, conical, rectangular, triangular, or polyhedral pattern. Such desired pattern can be spun by providing a field to the fiber, for instance using a voice coil from a loudspeaker during fiber spinning (see, e.g., FIG. 1).

Where desired, the fibers are arrayed onto a substrate in a defined orientation. Preferably, the locations onto which the fibers are deposited are addressable, allowing the biomolecule to be re-visited, detected and analyzed by correlating to the location. The location can, for example, be stored in a computer, and easily re-accessed. The substrate can also be used to contact the fiber with one or more reactive solutions, allowing the handling of the fiber through various chemical processes. The substrate can have a specific orientation and locating marks such that it could be placed in a reader which can find specific locations. The substrate can be a rotating planar substrate, or a planar substrate that is moved in another manner such as a linear translation (See FIG. 2). In some cases, the substrate is not planar, but can be, for instance, a rotating cylinder onto which the fiber is deposited. The substrate can be in the form of a disk, and be addressed in a manner analogous to a compact disc (CD) or a computer hard drive. The fibers from electrospinning or from other fiber spinning methods can be deposited onto a spinning planar substrate radially in a manner that is compatible with reading the information as in a CD, or computer hard drive.

The fiber arrays may be incorporated into a structure that provides for ease of analysis, high throughput, or other advantages, such as in a microtiter plate, waveguides, and the like. Such setup is also referred to herein as an “array of arrays.” For example, the subject arrays can be incorporated into another array such as zero-mode waveguides (see e.g., U.S. Pat. Nos. 7,170,050, 7,013,054, 6,917,726) microtiter or multi-well plate wherein each micro well of the plate contains a subject array of optical confinements. Typically, such multi-well plates comprise multiple reaction vessels or wells, e.g., in a 48 well, 96 well, 384 well or 1536 well format.

In some cases, it is desirable for the fiber material to be cross-linked. Cross-links are bonds linking one polymer chain to another. Cross-links are the characteristic property of thermosetting plastic materials. Cross-links between polymer chains can create a 3-dimensional network structure. The network structure can create a material that can be swollen with a compatible solvent, but will not be dissolved. The amount of cross-linking, or cross-link density can be controlled to form either a loose network or a tight network of crosslinks. The cross-link density can be characterized by a molecular weight between cross-links. A high molecular weight between cross links corresponds to a low cross link density and a loose network, and a low molecular weight between crosslinks corresponds to a high cross-link density and a tight network. A loose network will allow more swelling with solvent, allowing, for instance, a binding molecule to penetrate the fiber material to interact with the isolated biomolecule. A tight network will swell less, resulting in more effective restraint of the isolated biomolecule. The level of cross-linking can be controlled in order to allow the desired amount of access and the desired level of restraint of the biomolecule. The swollen network of cross-linked polymer is often referred to as a gel. Thus, the cross liking and swelling of the fiber can result in the creation of a gel which can be used as one of skill in the art would use a gel, for example for electrophoresis, or for separating molecules by size. In addition, cross-linking can inhibit close packing of polymer chains, preventing the formation of crystalline regions. The restricted molecular mobility of a crosslinked structure can limit the extension of the polymer material under loading. Cross-links can be formed by chemical reactions that are initiated, for example, by heat, light, electrons, or pressure. Cross-links can also be formed by the mixing and polymerization of an unpolymerized or partially polymerized resin, usually containing multifunctional components.

In many cases, cross-linking is irreversible, creating a thermosetting material. For example, electron beams can be used to cross-link polyethylene. Polymers can also be cross-linked with the use of a peroxide or by addition of a cross-linking agent such as vinylsilane along with a catalyst. In some cases, cross-links can be created by physical rather than covalent chemical links, for example by phase separation of domains of a block copolymer such as styrene butadiene, or, for example, by the formation of microcrystalline regions that act as cross links. In some cases, these physical cross-links can be broken and formed reversibly, for instance by heating and cooling. Using these methods, the level of cross-linking can be controlled by, for instance the amount of UV or electron radiation, the level of cross-linking additives, and the molecular weight of the monomeric units that are polymerized into the network.

It may be desired for the fiber material precursor be initially un-cross-linked or partially cross-linked in order to enhance the liquid nature of the precursor to facilitate processing and dissolution of the biomolecule, although in some embodiments it may be desirable to initiate the cross-linking process during the precursor stage. In some embodiments, the fiber material will be cross linked during fiber formation. In other embodiments, the fiber will be cross-linked subsequent to fiber formation. In some embodiments, the cross-linking will occur both during and subsequent to fiber formation. Cross-linking can be performed, for example, by UV or electron beam irradiation of the polymer precursor at the point of fiber formation, for example as it is coming out of the die of an extrusion or as it is ejected from an electrospinning tip. In some cases, the rate of cross-linking will be slow compared to the rate of fiber formation, in which case, it is desirable to crosslink the fiber after it is formed, for instance, exposing the fiber to UV or electrons after the fiber is deposited onto the substrate.

In order to obtain isolated biomolecules in the fibers, typically, the biomolecule is preferably soluble in, or molecularly compatible with the precursor to the fiber material. Thus, in the three types of processes described above, the biomolecule should be soluble in either: 1) the molten polymer, 2) the monomer solution, or 3) the polymer solution in solvent. Different biomolecules will have different solubilities. Intrinsic solubility is sometimes defined as the maximum concentration to which a solution can be prepared with a specific solute and solvent. A solute is the dissolved agent, here the solute is the biomolecule. Solubility depends on the nature of the biomolecule and of the fiber precursor as well as other factors such as the concentration, temperature, pressure, and pH. Solubility as used herein is a relative rather than absolute term. Complete solubility is the state in which the biomolecule solute is completely mixed with the fiber precursor material on the molecular level. It is usually desirable that the biomolecule be very soluble in the fiber material precursor in order to obtain a fiber with isolated biomolecules. The biomolecules should be soluble in the sense that they are well mixed with the fiber material precursor, but is usually not required that the biomolecules be soluble at high concentrations or have high intrinsic solubility. One skilled in the art can readily ascertain solubility by assessing the contribution of dispersion, polar, and hydrogen bonding components of the solvent and solute, appropriately soluble components and other factors. Those skilled in the art would be able to determine a biomolecule—fiber precursor material combination with the requisite level of solubility.

A fiber forming process which includes a solvent as a component of the polymer precursor can be a versatile system for obtaining the desired solubility of the biomolecule in the fiber material precursor. Many biomolecules of interest, for example biopolymers such as nucleic acids, polypeptides, and carbohydrates, are polar molecules that are dissolved most readily in polar systems. The fiber forming process in which the polymer is dissolved in a solvent, allows for the use of solvents such as water in which the biomolecules are soluble. While water is a good solvent for these materials, other polar solvents, alone, or in combination can be useful. Polar solvents useful for dissolving the biomolecule in the fiber precursor solution include, for example, polar amides such as formamide and dimethylformamide; sulfur-containing compounds such as dimethyl sulfoxide and sulfolane; imidazolidones such as 1,3-dimethyl-2-imidazolidone; ethers such as dioxane, 1,2-dimethoxyethane, and diglyme; halohydrocarbons such as dichloromethane, chloroform, and 1,2-dichloroethane; acid anhydrides such as acetic anhydride and propionic anhydride; and carboxylic acids such as acetic acid, trifluoroacetic acid, and propionic acid, and mixtures thereof.

The solubility of a biopolymer can be dependent on the molecular weight of the polymer. The molecular weight of a polymer is related to how long the polymer is and the number of repeat units in the polymer chain. In general, a polymer becomes more difficult to solubilize the higher the molecular weight. This difficulty in solubilizing a biopolymer has both a thermodynamic and a kinetic component. Thermodynamic relating to whether the solubilization is overall energy favorable, and kinetics relating to the rate at which the polymer dissolves. In some embodiments, the addition of solubilizing components can be used to improve both the kinetics and thermodynamics of dissolution of the polymer. In some embodiments, the addition of a polyelectrolyte can be added to improve solubility. In some embodiments, for instance, a polymer with carboxylate groups is added along with a nucleic acid such as DNA in order to improve its solubility in the fiber material precursor. Suitable carboxylate containing polymers for aiding solubility include anionic poly(amino acids) such as polyaspartate, polyglutamate, polyacrylic acid, and poly(acrylic acids) such as polyacrylic acid, polymethacrylic acid and their salts (see Tang, et al., Am. J. Physiol. Lung Cell Mol. Physiol. 289, L599-L605, 2005).

Any biomolecules can be isolated or embedded in the subject fibers. Non-limiting examples of biomolecules include sugars, fatty acids, steroids, triglycerides, lipids, and isoprenoids. Biomolecules of particular interest in the present invention are biopolymers including without limitation nucleic acids, polypeptides, and polysaccharides, and polymers related to or derived from these biopolymers.

“Nucleic acid” or “oligonucleotide” or “polynucleotides” are used interchangeably to mean a polymeric form of nucleotides of any length, either deoxyribonucleotides or ribonucleotides, or analogs thereof. Polynucleotides may have any three-dimensional structure, and may perform any function, known or unknown. A nucleic acid of the present invention will generally contain phosphodiester bonds, although in some cases, nucleic acid analogs are included that may have alternate backbones, comprising, for example, phosphoramide, phosphorothioate, phosphorodithioate, O-methylphophoroamidite linkages, and peptide nucleic acid backbones and linkages. Other analog nucleic acids include those with bicyclic structures including locked nucleic acids, (Koshkin et al., J. Am. Chem. Soc. 120:13252 3 (1998)); positive backbones (Denpcy et al., Proc. Natl. Acad. Sci. USA 92:6097 (1995)); non-ionic backbones (U.S. Pat. Nos. 5,386,023, 5,637,684) and non-ribose backbones. Nucleic acids containing one or more carbocyclic sugars are also included within the definition of nucleic acids. Nucleic acids can be DNA, RNA or a hybrid of both. They can be synthetic, recombinantly produced, or directly extracted from an organism's genome.

A “polypeptide” or “peptide” refers to a biopolymer comprised of linked amino acids, thus the terms polypeptide and poly(amino acid) are used interchangeably. A protein is a polypeptide, and is sometimes distinguished from a simple polypeptide in that a protein is relatively large and often has a function. As used herein, there is no particular dividing line between a protein and a polypeptide, and both can be biopolymers as used herein. Proteins are important biopolymers, acting as catalysts (enzymes), in signal transduction, and as structural proteins. The 3-dimensional structure of the protein can be important to its function. One embodiment of the invention is an isolated protein in a fiber with its 3-dimensional structure is related to its 3-dimensional structure as an active protein. In another embodiment, the isolated protein in the fiber is elongated from its active 3-dimensional structure.

A polysaccharide as used herein is a biopolymer made up of sugars (monosaccharides) joined together by glycosidic linkages. Polysaccharides can act as energy storage, for example in form of starch, and can have structural functions, for example by cellulose and chitin. Starch comprises a glucose polymer in which the glucopyranose units are bonded by alpha-linkages. Starch is made up of a mixture of Amylose and Amylopectin. Amylose consists of a linear chain of several hundred glucose molecules and Amylopectin is a branched molecule made of several thousand glucose units. Cellulose is a polymer made with repeated glucose units bonded together by beta-linkages that forms a major part of the structural component of plants.

As used herein with respect to linked units of a polymer, “linked” or “linkage” means two entities are bound to one another by any physicochemical means. Any linkage known to those of ordinary skill in the art, covalent or non-covalent, is embraced. Such linkages are well known to those of ordinary skill in the art. Natural linkages, which are those ordinarily found in nature connecting the individual units of a particular polymer, are most common. Natural linkages include, for instance, amide, ester and thioester linkages. The individual units of a polymer analyzed by the methods of the invention may be linked, however, by synthetic or modified linkages. Polymers where the units are linked by covalent bonds will be most common.

The biopolymers of the present invention can include combinations of nucleic acids, polypeptides, and polysacchrides. For example proteoglycans and glycosaminoglycans have polysaccharide backbones containing amino acid substituents, and glycoproteins have carbohydrates attached to proteins.

The biopolymers may be native or naturally-occurring polymers which occur in nature or non-naturally occurring polymers which do not exist in nature. The biopolymers may include at least a portion of a naturally occurring polymer. The biopolymers can be isolated or synthesized de novo. For example, the biopolymers can be isolated from natural sources e.g. purified, as by cleavage and gel separation or may be synthesized e.g., (i) amplified in vitro by, for example, polymerase chain reaction (PCR); (ii) synthesized by, for example, chemical synthesis; or (iii) recombinantly produced by cloning, etc. The isolated biopolymers may be genomic DNA from, for example, eukaryotes, prokaryotes, or archae, or may be RNA. The RNA may be, for instance, messenger RNA, transfer RNA, or Ribosomal RNA.

In some embodiments, the subject biomolecules are labeled. Such labeling can be effected by interacting with a compound, ligand, or binding agent to produce a signal characteristic of that interaction. In the case of biopolymer, at least one repeating unit thereof is typically capable of interacting with a compound, ligand, or binding agent to produce the signal characteristic of that interaction. If a unit of a biopolymer can undergo that interaction to produce a characteristic signal, then the biopolymer is said to be intrinsically labeled. It is not necessary that an extrinsic label be added to the biopolymer. If a non-native molecule, however, must be attached to the individual unit of the biopolymer to generate the interaction producing the characteristic signal, then the biopolymer is said to be extrinsically labeled. The “label” may be, for example, light emitting, energy accepting, fluorescent, radioactive, quenching, scattering, or provide electron or neutron contrast.

The biomolecules can be treated or processed in other ways before, during, or after fiber formation. For instance, the biomolecules can be purified and isolated. For example, for genomic DNA, the DNA can be separated from the histones and other components prior to elongation. In some embodiments, a single microfluidic device can perform the purification of the biomolecule and the formation of the fiber. The biomolecules can be treated with binding agents before, during or after fiber formation. In some embodiments, the binding agents are labeled. For example, in some embodiments, binding agents with labels that bind to specific locations on a biomolecule can be used to provide sequence or structure information about a biopolymer. In some cases, the labeled binding agents can be added prior to fiber formation. The binding agent can remain bound to the biopolymer during fiber formation, and is then detected in the formed fiber. This approach can be advantageous because it allows binding while the biopolymer is in a liquid medium. In some cases the biding agent will affect the way the biopolymer orients and elongates during fiber formation providing further structural information about the biopolymer. In other embodiments, the biopolymer is first isolated and fixed in the fiber, and the labeled binding agent is only added subsequent to fiber formation. This embodiment allows the biopolymer to be observed prior to and after labeling, and allows the binding process to be observed. In one embodiment, a substrate with a deposited fiber containing a plurality of isolated biomolecules is treated with a solution containing a plurality of labeled binding agents, and the sequence and/or structure of the isolated biomolecules is detected and/or determined by observing the binding of the labeled binding agents. In some embodiments, the binding agents are proteins that bind to specific molecules. In some embodiments, the binding agents are immunoglobulins. In some embodiments the binding agents bind to specific DNA sequences. In some embodiments the binding agents are transcription factors including but not limited to TFIIA, TFIIB, TFIID, TFIIE, TFIIF, TFIIH, and translation initiation factors including but not limited to elF1, elF1A, elF2, elF2A, elF2B, elF3, elF3A, elF4A, elF4B, elF4E, elF4F, elF4G, elF4H, elF5, elF5B, and Ded1.

Labels useful for electron microscopy include, for example, heavy metal labeled binding proteins such as immunoglobulins, avidin, streptavidin, digoxigenin, and DNA and RNA binding proteins. The proteins can be labeled with gold and gold clusters, formation of immunogold conjugates (EMSciences) (see e.g. G. Griffiths, “Fine Structure Immunocytochemistry.” Springer Verlag, Heidelberg & Berlin (1993) and Science 236, 450-453, 1987); Iron, for example via ferritin conjugates; Lead, for example via lead citrate, polysaccharide conjugates, see (J. Histochem. Cytochem. Vol. 23(3), 169-173, 1975; Iridium and cobalt via CO clusters; as well as other electron dense atoms such as silver; mercury, uranium, platinum, rhodium, ruthenium, palladium, copper, and selenium. The biopolymers may be functionalized with heavy metals via functional groups on the biopolymer. For example, the SH groups on a poly peptide can be reacted with, for example mercury chloride, ethyl-mercury phosphate, and osmium pent amine, iridium pentaminc to label the protein with a heavy metal atom.

Gold particles can be used to label nucleic acids such as DNA. The labeled DNA can be detected by electrons or light, and different size gold particles can be differentiated because the scatter light of different wavelengths (see Park, et al. U.S. Pat. No. 7,169,556).

Many naturally occurring units of a biopolymer are light emitting compounds or quenchers. For instance, nucleotides of native nucleic acid molecules have distinct absorption spectra, e.g., A, G, T, C, and U have absorption maximums at 259 nm, 252 nm, 267 nm, 271 nm, and 258 nm respectively. Modified units which include intrinsic labels may also be incorporated into biopolymers. A nucleic acid molecule may include, for example, any of the following modified nucleotide units which have the characteristic energy emission patterns of a light emitting compound or a quenching compound: 2,4-dithiouracil, 2,4-Diselenouracil, hypoxanthine, mercaptopurine, 2-aminopurine, and selenopurine.

The type of extrinsic label selected to detect the isolated biomolecule with light will depend on a variety of factors, including the nature of the analysis being conducted, the type of the agent, the fiber material, and the type of biopolymer. Extrinsic label compounds include but are not limited to light emitting compounds, quenching compounds, radioactive compounds, spin labels, and heavy metal compounds. The label should be sterically compatible and chemically compatible with the units of the boipolymer being analyzed.

A “light emissive compound” as used herein is a compound that emits light in response to irradiation with light of a particular wavelength. These compounds are capable of absorbing and emitting light through phosphorescence, chemiluminescence, luminescence, polarized fluorescence, scintillators or, more preferably, fluorescence. The particular light emissive compound selected will depend on a variety of factors which are discussed in greater detail below. Light emissive compounds have been described extensively in the literature. For example, Haugland, R. P., Handbook of Fluorescent Probes and Research Chemicals, 6th edition, Molecular Probes, Inc., 1996, which is hereby incorporated by reference provides a description of light emitting compounds.

Radioactive compounds can be used to label biomolecules embedded in the subject fibers. Radioactive compounds comprise substances which emit alpha, beta, or gamma nuclear radiation. Alpha rays are positively charged particles of mass number 4 and slightly deflected by electrical and magnetic fields. Beta rays are negatively charged electrons and are strongly deflected by electrical and magnetic fields. Gamma rays are high energy electromagnetic radiation (photons) and are undeflected by electrical and magnetic fields.

Fluorescent compounds can also be used to label the subject biomolecules. Fluorescent compounds such as dyes comprise molecules having a chain of several conjugated double bonds. The absorption and emission wavelengths of a dye are approximately proportional to the number of carbon atoms in the conjugated chain. A preferred fluorescent compound is “Cy-3” (Biological Detection Systems, Pittsburgh, Pa.). Other preferred fluorescent compounds useful according to the invention include but are not limited to fluorescein isothiocyanate (“FITC”), Texas red, tetramethylrhodamine isothiocyanate (“TRITC”), 4,4-difluoro-4-bora-3a, and 4a-diaza-s-indacene (“BODIPY”). In some cases the fluorescent dye interacalates into the a DNA helix.

Chemiluminescent compounds are compounds which produce luminescence due to a chemical reaction. Phosphorescent compounds are compounds which exhibit delayed luminescence as a result of the absorption of radiation. Luminescence is a non-thermal emission of electromagnetic radiation by a material upon excitation. These compounds are well known in the art and are available from a variety of sources.

In one embodiment of the invention the light emissive compound includes a donor or an acceptor fluorophore. A fluorophore as used herein comprises a molecule capable of absorbing light at one wavelength and emitting light at another wavelength. Fluorophores can be photochemically promoted to an excited state, or higher energy level, by irradiating them with light. Excitation wavelengths are generally in the UV, blue, or green regions of the spectrum. The fluorophores typically remain in the excited state for a very short period of time before releasing their energy and returning to the ground state. Those fluorophores that dissipate their energy as emitted light are generally considered donor fluorophores. The wavelength distribution of the outgoing photons forms the emission spectrum, which peaks at longer wavelengths (lower energies) than the excitation spectrum, but is equally characteristic for a particular fluorophore. A donor fluorophore is a fluorophore which is capable of transferring its fluorescent energy to an acceptor molecule in close proximity. Conversely, an acceptor fluorophore is a fluorophore that can accept energy from a donor at close proximity. An acceptor of a donor fluorophore does not have to be a fluorophore. It may be non-fluorescent.

Where desired, the donor and/or the acceptor can comprise a quenching source which is capable of altering a property of a light emitting source. The property which is altered can include intensity fluorescence lifetime, spectra, fluorescence, or phosphorescence. In some cases, a quenching source can be attached to a binding agent that binds to specific portions of a biomolecule such that the detection of quenching can be used to detect the presence or absence of a specific structure or portion of a biopolymer, for instance, the sequence of the biopolymer.

Extrinsic labels can be added to the biopolymer by any means known in the art. For example, the labels may be attached directly to the biopolymer or attached to a linker that is attached to the biopolymer. For instance, fluorophores have been directly incorporated into nucleic acids by chemical means but have also been introduced into nucleic acids through active amino or thio groups into a nucleic acid (see Proudnikov and Mirabekov, Nucleic. Acids Research, 24: 4535-4532, 1996). Modified units which can easily be chemically derivatized or which include linkers can be incorporated into the biopolymer to enhance this process. An extensive description of modification procedures which can be performed on the biopolymer, the linker and/or the extrinsic label in order to prepare a bioconjugate can be found in Hermanson, G. T., Bioconjugate Techniques, Academic Press, Inc., San Diego, 1996. The extrinsic labels can also be attached to a binding agent which binds, for instance to a particular location within a biopolymer. For example, the label can be attached to a transcription factor that binds to a particular portion of a nucleic acid. The extrinsic labels can also be added to the biopolymers by weaker binding interactions such as intercalation. Intercalating dyes are dyes that exhibit enhanced fluorescence when they bind with the nucleic acid. With double-stranded nucleic acids, the dyes can bind within the helix. The dyes can also show an enhanced fluorescence upon binding to single stranded nucleic acids. Examples of dyes that can exhibit enhanced fluorescence when associated with nucleic acid molecules include bisbenzimide or indole-derived dyes (e.g. Hoechst 33342, Hoechst 33258 and 4′,6-diamidino-2-phenylindole), phenanthridinium dyes (e.g. ethidium bromide and propidium iodide) and cyanine dyes (e.g. PicoGreen, YOYO, TOTO, PicoGreen, SYBR Green, and SYBR Gold).

Methods are known for the direct chemical labeling of DNA. One of the methods is based on the introduction of aldehyde groups by partial depurination of DNA. Fluorescent labels with an attached hydrazine group are efficiently coupled with the aldehyde groups. The reaction of cytosine with bisulfite in the presence of an excess of an amine fluorophore leads to transamination at the N-4 position. Reaction conditions such as pH, amine fluorophore concentration, and incubation time and temperature affect the yield of products formed.

Extrinsic labels can be attached to biopolymers or other materials by any mechanism known in the art. For instance, functional groups which are reactive with various light emissive groups include, but are not limited to, (functional group: reactive group of light emissive compound) activated ester:amines or anilines; acyl azide:amines or anilines; acyl halide:amines, anilines, alcohols or phenols; acyl nitrile:alcohols or phenols; aldehyde:amines or anilines; alkyl halide:amines, anilines, alcohols, phenols or thiols; alkyl sulfonate:thiols, alcohols or phenols; anhydride:alcohols, phenols, amines or anilines; aryl halide:thiols; aziridine:thiols or thioethers; carboxylic acid:amines, anilines, alcohols or alkyl halides; diazoalkane:carboxylic acids; epoxide:thiols; haloacetamide:thiols; halotriazine:amines, anilines or phenols; hydrazine:aldehydes or ketones; hydroxyamnine:aldehydes or ketones; imido ester:amines or anilines; isocyanate:amines or anilines; and isothiocyanate:amines or anilines.

The subject fibers can be provided with a detection system capable of detecting the embedded biomolecule(s) and/or monitoring interactions between reactants even at the single-molecule level. The detection system can detect, for example, optical signals, electrons, X-rays, or neutrons. A suitable optical system achieves these functions by first generating and transmitting an incident wavelength to the biomolecules contained in the fibers, followed by collecting and analyzing the optical signals from the biomolecules and/or reactants bound directly or indirectly to the embedded biomolecules. Such systems may employ an optical train that can direct signals to different locations of an array of fibers and simultaneously detect multiple different optical signals from each of multiple different fibers. In particular, the optical trains typically include optical gratings or wedge prisms to simultaneously direct and separate signals having differing spectral characteristics on an array based detector, e.g., a CCD.

The optical system applicable for the present invention comprises at least two elements, namely an excitation source and a photon detector. The excitation source generates and transmits incident light used to optically excite the biomolecules and/or reactants contained in the fiber. Depending on the intended application, the source of the incident light can be a laser, laser diode, a light-emitting diode (LED), a ultra-violet light bulb, and/or a white light source. Where desired, more than one source can be employed simultaneously. The use of multiple sources is particularly desirable in applications that employ multiple different reactant compounds having differing excitation spectra, consequently allowing detection of more than one fluorescent signal to track the interactions of more than one or one type of molecules simultaneously. A wide variety of photon detectors are available in the art. Representative detectors include but are not limited to optical reader, high-efficiency photon detection system, photodiode (e.g. avalanche photo diodes (APD)), camera, charge couple device (CCD), electron-multiplying charge-coupled device (EMCCD), intensified charge coupled device (ICCD), and confocal microscope equipped with any of the foregoing detectors.

The subject optical system may also include an optical transmission element whose function can be manifold. First, it collects and/or directs the incident wavelength to the fiber containing the biomolecules and/or other reactants. Second, it transmits and/or directs the optical signals emitted from the reactants inside the fiber to the photon detector. Third, it may select and/or modify the optical properties of the incident wavelengths or the emitted wavelengths from the biomolecules and/or reactants. Illustrative examples of such element are diffraction gratings, arrayed waveguide gratings (AWG), optic fibers, optical switches, mirrors, lenses (including microlens and nanolens), collimators. Other examples include optical attenuators, polarization filters (e.g., dichroic filter), wavelength filters (low-pass, band-pass, or high-pass), wave-plates, and delay lines. In some embodiments, the optical transmission element can be planar waveguides in optical communication with the arrayed fibers.

The optical transmission element suitable for use in the present invention encompasses a variety of optical devices that channel light from one location to another in either an altered or unaltered state. Non-limiting examples of such optical transmission devices include optical fibers, diffraction gratings, arrayed waveguide gratings (AWG), optical switches, mirrors, (including dichroic mirrors), lenses (including microlens and nanolens), collimators, filters, prisms, and any other devices that guide the transmission of light through proper refractive indices and geometries.

The electron microscope systems suitable for use in the present invention include but are not limited to transmission electron microscopy (TEM), scanning electron microscopy (SEM), and reflection electron microscopy (REM). TEM involves a high voltage electron beam emitted by a cathode and formed by magnetic lenses. The electron beam that has been partially transmitted through a thin specimen carries information about the inner structure of the specimen. The spatial variation in this information (the “image”) is then magnified by magnetic lenses where it is recorded by hitting a fluorescent screen, photographic plate, or other light sensitive sensor such as a CCD (charge-coupled device) camera. The image detected by the CCD may be displayed in real time on a monitor or computer. Resolution of the high-resolution TEM (HRTEM) can be limited by spherical aberration and chromatic aberration, but a new generation of aberration correctors has been able to overcome spherical aberration. High resolution TEM (HRTEM) with software correction of spherical aberration has allowed the production of images with sufficient resolution to show carbon atoms in diamond separated by only 0.089 nanometers and atoms in silicon at 0.078 nanometers at magnifications of 50 million times. In some embodiments, the thin fibers are deposited over an opening allowing TEM images of the isolated biomolecules to be detected.

SEM produces images by detecting secondary electrons which are emitted from the surface due to excitation by the primary electron beam. In the SEM, the electron beam is rastered across the sample, with detectors building up an image by mapping the detected signals with beam position. Generally, the TEM resolution is about an order of magnitude better than the SEM resolution, however, because the SEM image relies on surface processes rather than transmission it is able to image bulk samples and has a much greater depth of view, and so can produce images that are a good representation of the 3D structure of the sample. REM, like TEM, involves electron beams incident on a surface, but instead of using the transmission (TEM) or secondary electrons (SEM), the reflected beam is detected.

### Uses of the Subject Fibers, Systems and Other Devices:

The subject devices including fibers and the associated detection systems provide an effective means for isolating, manipulating, and analyzing individual biomolecules including but not limited to biopolymers. The fibers of the present invention are an effective tool with which a single biomolecule can be trapped, stored, fixed, manipulated for any subsequent analyses. The fibers and the associated detection systems are also useful for conducting and monitoring chemical reactions whether in real time or otherwise. In particular, the subject device and detection/monitoring methods may be used in a wide variety of circumstances including analysis of biochemical and biological reactions for diagnostic and research applications. For example, the present invention can be applied in the elucidation of nucleic acid sequences for research applications, and particularly in sequencing individual human genomes as part of preventive medicine, rapid hypothesis testing for genotype-phenotype associations, in vitro and in situ gene-expression profiling at all stages in the development of a multi-cellular organism, determining comprehensive mutation sets for individual clones and profiling in various diseases or disease stages. Other applications include measuring enzyme kinetics, and identifying specific interactions between target molecules and candidate modulators of the target molecules. Further applications involve screening factors (e.g., transcription factors) involved in regulating gene expression or factors (e.g., translation initiation factors) that regulate protein expression. The present invention can be used in conjunction with enzyme linked immunosorbent assay (ELISA) or fluorescent in-situ hybridization (FISH) analyses.

In certain embodiments, the subject devices and methods allow high-throughput single-molecule analysis. Single-molecule analysis provides several compelling advantages over conventional approaches to studying biological events. First, the analysis provides information on individual molecules whose properties are hidden in the statistically averaged information that is recorded by ordinary ensemble measurement techniques. In addition, because the analysis can be multiplexed, it is conducive to high-throughput implementation, requires smaller amounts of reagent(s), and takes advantage of the high bandwidth of optical systems such as modern avalanche photodiodes for extremely rapid data collection. Moreover, because single-molecule counting automatically generates a degree of immunity to illumination and light collection fluctuations, single-molecule analysis can provide greater accuracy in measuring quantities of material than bulk fluorescence or light-scattering techniques. As such, single-molecule analysis greatly improves the efficiency and accuracy in determining protein-protein interaction, nucleic-protein interaction, genotyping, gene expression profiling, DNA sequencing, nucleotide polymorphism detection, pathogen detection, protein expression profiling, and drug screening.

Accordingly, in one embodiment, the present invention provides a method of isolating a biomolecule comprising the steps of mixing a biomolecule into a fiber forming material; and forming a fiber that comprises the biomolecule embedded therein, thereby isolating said biomolecule. Any of the aforementioned methods for fabricating the subject fibers can be used to isolate a biomolecule.

The subject biomolecules are generally isolated such that the individual molecules are substantially devoid of at least some of the other components that may also be present where the substance or a similar substance naturally occurs or is initially obtained from. Isolated biomolecules are typically independently observable by one or more detection means. Preferably, the biomolecules are isolated such that they can be detectable without the interference of other biomolecules in the fiber via a given detection method. In one aspect, isolation means that there is only one molecule within a given volume element of the fiber. For instance, if the chain axis of the fiber is considered as the z axis and the cross sectional dimensions as the x and y axes, a molecule is isolated if one detects one molecule while moving along the z axis of the fiber. That is, there lacks substantial overlap of molecules along the z axis. In other embodiments, there may be some overlap of the isolated molecules along the z axis of the fiber as long as there is a separation between the isolated molecules in the x and or y dimensions such that the biomolecules are independently observable. Practically, the space between molecules may be larger than that minimally necessary for the biomolecule to be isolated. For example, where an isolated biomolecule is observable by TEM, it may be too close to a neighboring biomolecule to be distinguished by optical means such as fluorescence.

The distance between molecules can be controlled by controlling the concentration of the biomolecules within the fiber material. The concentration range which will produce isolated biomolecules in the fiber will also depend on the size of the molecule, the orientation of the molecule, the thickness of the fiber and in some cases, on the amount of elongation of the biomolecule. As a starting point determining the concentration to use, a calculation can be made of the volume of a cross section of fiber material that contains a single biomolecule. For instance, for a biomolecule with a diameter of 2 nanometers in a fiber with a diameter of 200 nanometers, the volume of a cross section of a round fiber containing only that biomolecule is 6.3 E-20 liters. This corresponds to a concentration of 2.6 E-5 moles/liter. This calculation provides an estimate of the range of concentrations for an isolated biomolecule within the fiber. Where the concentration is lowered, a higher fraction of the biomolecules will be isolated within the fiber. It would be understood by those skilled in the art that the molecules in a solution are not perfectly spaced from one another, and there is generally a randomness to the distribution of the dissolved molecules within the volume. This randomness will result in a probability distribution for obtaining isolated biomolecules. Where a biomolecule is elongated along the axis of the fiber, the concentration range for obtaining isolated biomolecules will generally be lower than for the same molecule in its non-elongated configuration. A lower concentration of biomolecules will generally be required for a biomolecule that is larger, than for a biomolecule that is larger. As the fiber is made thicker, a lower concentration of biomolecules will generally be required to obtain isolated biomolecules than where the fiber is thinner.

In some cases, it is advantageous to control the concentration such that, on average, one biomolecule is present in a given length of fiber. Thus, for example, for an elongated biopolymer that is expected to be about 16 micrometer in length, it may be desired to have, on average, one biomolecule every 500 micrometers of fiber. In some embodiments, the concentration is controlled to provide, on average, one biomolecule for every 10, 100, 1,000, or 10,000 nanometers. In some embodiments the concentration is controlled to provide, on average, one biomolecule for every 10, 100, 1,000, or 10,000, micrometers. In some embodiments, the concentration is controlled to provide, on average, one biomolecule for 10, 100, 1,000, 10,000 millimeters.

The subject methods can generate isolated and fixed biomolecule. The term “fixed” as used herein means that the biomolecule is constrained in its movement. One aspect of fixation is constraint of the translational movement of a biomolecule. The fiber material will constrain the biomolecule from translational movement with respect to the fiber. If the fiber is deposited on a substrate the translational movement can be constrained with respect to the substrate, and where the molecule is detected, the movement can be constrained with respect to the detector. Another aspect of fixation is constraint of rotational movement of the biomolecule. In some cases, the biomolecule may be constrained translationally, but free to rotate in that position. In other cases, the fiber material can be used to constrain the molecule from rotation as well as translation. It will be understood that a biomolecule will have several axes of rotation, and that the fiber material can be used to constrain some, but not other axes of rotation. Another aspect of fixation is the constraint of intramolecular motions. Even where a biomolecule is constrained from molecular rotation, the various components of the molecule will be undergoing translation, rotation, and vibration. For instance, a fluorescent dye attached to a biomolecule may have an aromatic ring structure, and even where the molecule is held is place, the aromatic ring structure will be freely rotating. In some cases, it may be desirable for the dye structure to be allowed to freely rotate, whereas in other cases, for example to obtain information on orientation, constraint of the dye structure is desired. The type of fiber material and the temperature, for example, can be used to control the constraint the intramolecular motions of the biomolecule.

In some embodiments, the fiber material can be made more rigid in order to constrain the biomolecule. In some cases, a cross-linked network can be used to control the constraint of the biomolecule. A more highly cross-linked network will provide to more constraint, a more loosely cross-linked network will provide less constraint. In addition, low molecular weight compounds can be included in order to allow more movement of molecules within the fiber. Cross-linked materials with low molecular weight compounds within them are commonly referred to as gels, and the control of gel properties is well known in the art.

In some embodiments the amount of fixation on an isolated biomolecule can be changed over time. For instance, it may be desired to first rigidly constrain an elongated biomolecule within a fiber for observation, then subsequently to allow the constrained biomolecule more freedom, for example, to allow a binding agent to bind to it. In another embodiment, the an elongated, rigidly constrained biopolymer could later be allowed to move such that it adopts its unconstrained configuration, allowing observation of the molecular dynamics and formation of 3-dimensional structure. In another embodiment, the amount of constraint on the biomolecule can be increased at a later time. The amount of constraint can be lowered for example, by reducing the crosslink density, by adding solvents to the fiber material, or by raising the temperature to increase mobility. The cross-link density can be lowered, for instance with the inclusion of reversible cross-links as described above. The amount of constraint can be increased on an isolated biomolecule, for instance, by increasing the cross-link density, by removing solvents from the fiber material, or by lowering the temperature to reduce mobility.

The subject methods can produce isolated and elongated biomolecules including without limitation a variety of elongated biopolymers. An elongated biopolymer typically has a dimension which is longer than a dimension of a biopolymer in its un-elongated state. For many biopolymers, the un-elongated dimensions can be calculated by assuming that the biopolymer is in a random coil configuration. The random coil is a polymer conformation where the monomer subunits are oriented randomly while still being bonded to adjacent units. It is not one specific shape, but a statistical distribution of shapes for all the chains in a population of macromolecules. Many linear, unbranched homopolymers in solution, or above their melting temperatures approximate random coils. Even copolymers with monomers of unequal length will distribute in random coils if the subunits lack any specific interactions. The parts of branched polymers may also assume random coils. More complex polymers such as polypeptides, proteins, and some RNA and DNA molecules with various interacting chemical groups attached to their backbones, self-assemble into well-defined structures. Segments of proteins, and polypeptides that lack secondary structure, can generally be approximated as a random coil. The methods of calculating the random coil configurations of polymers are well known (see Flory, Principles of Polymer Chemistry, Cornell University Press, 1953; Flory, P. J., Statistical Mechanics of Chain Molecules, Wiley. 1969).

The dimensions of a biopolymer in its un-elongated state can typically be detected using spectroscopic techniques such as light scattering, neutron scattering, circular dichroism and nuclear magnetic resonance (NMR). The arrangement of the planar amide bonds results in a distinctive signal in circular dichroism. The chemical shift of amino acids in a random coil conformation is well known in nuclear magnetic resonance (NMR).

One measure of elongation of the biopolymer is the ratio of the largest dimension of the elongated biopolymer with the largest dimension of the biopolymer in its un-elongated state within the fiber material. The biopolymer is elongated if the ratio of the largest dimension of the elongated polymer is 2, 5, 10, 50, 100, or 1000 times the largest dimension of the polymer in its un-elongated state within the fiber material.

Another measure of elongation of the biopolymer is the ratio of the largest dimension of the elongated polymer with the largest dimension of the polymer in its random coil configuration. The biopolymer is elongated if the ratio of the largest dimension of the elongated polymer is 2, 5, 10, 50, 100, or 1000 times the largest dimension of the polymer in its random coil configuration.

In some embodiments, the isolated biopolymer is elongated such that the polymer chain axis is oriented parallel to the longitudinal axis of the fiber. For example, where the biopolymer is a nucleic acid, by orienting the polymer parallel with the longitudinal axis of the fiber, the sequence of the nucleic acid is held in order down the length of the fiber, allowing sequence and structure information to be ascertained by visualizing the labels bound to the biopolymer or by visualizing cutting patterns along the length of the biopolymer. It is not required that the complete length of the polymer be held parallel to the fiber axis, only that enough of the biopolymer is in such orientation that information about the sequence, structure, or properties of the biopolymer can be obtained by the orientation of the biopolymer. The oriented biopolymer may also be in multiple pieces within the fiber. In some cases, the process of elongation creates shear that will result in breakage of the biopolymer into two or more pieces during the process of alignment. In some cases, the frequency and location of the breaks in the biopolymer can provide information about the sequence, structure, and properties of the biopolymer. One advantage of aligning the isolated biopolymers within a fiber is that the process can be performed such that the pieces of the biopolymer will tend to stay in order even after breakage. It can be appreciated that where a significant amount of the shear and alignment occur while the biopolymer is in the fiber in a dilute solution, that even when it breaks, other biopolymer molecules will not be able to intervene, and the pieces of the broken biomolecule will remain together. This aspect of the invention is highly beneficial when analyzing a large number of molecules such as the DNA representing the genome or the RNA representing the transcriptome. Knowledge that the neighboring pieces are related can assist in compiling the sequences, structure, and property information.

In some cases, it can be desirable to elongate the biopolymers prior to fiber formation. It can be advantageous to elongate the biomolecules prior to fiber formation to enhance the elongation of the biomolecules in the fiber, and also to increase the amount to which any broken fragments maintain their order within the fiber. In one embodiment, the biopolymers are pre-elongated by being subjected to a flow field prior to the fiber formation process. The flow field can be created, for instance, in a fluidic device that is coupled to the fiber formation apparatus. In some embodiments, a microfluidic device and an electrospinning tip can be fabricated in the same device to allow both pre-elongation, and fiber spinning. It is known that polymers can be elongated in elongational flow fields (see Perkins et al., Single Polymer Dynamics in an Elongational Flow, Science, 276, 2016, 1997; and Smith et al., Response of Flexible Polymers to a Sudden Elongational Flow, Science, 281, 1335, 1998). In another embodiment, the biopolymer can be pre-elongated before fiber spinning by constraining the DNA to a narrow channel. It is known in the art that biomolecules can be elongated in by constraint into narrow channels (see, for example, Mannion et al., J., 90(12), 4538, 2006; Reccius, C. H.; Mannion et al., Phys. Rev. Lett., 95, 2005; Schwartz, U.S. Pat. Nos. 6,147,198 and 6,509,158). In another embodiment, the biomolecules can be elongated prior to fiber formation by passing the biopolymers through nanostructured obstacles. Stretching polymers by passage through nanostructures is known in the art (see, for example, Chan et al., U.S. Pat. Nos. 6,696,022, 6,762,059, and 6,927,065). Other methods of elongating the biopolymers such as gravity, electrophoresis, optical tweezers, molecular combing, and tethering can also be utilized with the present invention to produce fibers with elongated isolated biopolymers.

In some embodiments the biopolymer is treated before fiber formation to enhance the amount of elongation. For example, where the biopolymer is a protein, the internal cross-links within the protein, such as disulfide linkages can be broken, for example by a reducing agent, such that the protein is more amenable to elongation. In other embodiments, the biopolymer is treated such that it retains its 3-dimensional shape during fiber formation. For example, a biopolymer such as an enzyme may be internally cross-linked in order to preserve its 3-dimensional structure such that its activity remains intact in its isolated state within the fiber.

The biomolecules can also be elongated subsequent to fiber formation. The point of fiber formation generally means the point at which the precursor liquid, be it a melt, monomers, a solution or a combination, becomes a solid. While there is a general understanding of what constitutes a liquid and a solid, the point at which the system goes from one state to the other is not a precise quantity. For the purposes of carrying out the invention, when, exactly, the fiber is formed is not critical. After the fiber is formed, continued elongation and manipulation of the elongated biomolecule in it can occur. In one embodiment, the fiber is further stretched after it is solid to elongate or further elongate the biomolecule in the fiber. Where the fiber has been deposited on a surface, portions of the fiber can be moved, resulting in elongation, and reorientation of the direction of the fiber. In some cases, the fibers of the present invention are very thin, for example less than 100 nanometers in diameter. For these types of fibers, atomic force microscopy (AFM) probes can be used to distort the fibers to orient and further elongate the molecules. In addition to elongation subsequent to deposition, the fibers can also be deformed in ways such as being stretched or flattened to change the shape or orientation of the fiber, or to distort or elongate the embedded biomolecule. Subsequent processing can involve thinning of the fiber, for example by etching of the fiber after deposition. Methods of etching materials using a plasma is well known in the art. The fibers can, for example, be etched using an oxygen plasma, making the fibers thinner, and in some cases, enhancing the observation and characterization of the embedded biomolecule.

In some embodiments of the invention, biopolymers such as nucleic acids are cleaved with a sequence-specific endonuclease, or restriction enzyme. A restriction enzyme is an enzyme will cut a nucleic acid molecule only where the nucleic acid has a specific set of bases. The use of restriction enzymes to cut elongated DNA in order to obtain sequence information is known in the art (see, for example, Schwartz, et al. U.S. Pat. Nos. 6,221,592, 6,509,158, and 6,147,198). In some embodiments, the restriction enzyme cuts the nucleic acid after fiber formation, in other embodiments, the restriction enzyme cuts the nucleic acid prior to fiber formation. In one embodiment, the nucleic acid is elongated in fluid prior to fiber formation, and the restriction enzyme cuts the elongated nucleic acid under flow. The flow can be maintained such that the cut pieces of the nucleic acid remain in sequential order after being cut, and thus, the nucleic acid pieces end up in order in the produced fiber. In some cases, the enzyme and nucleic acid can be processed together, with the enzyme in an inactivated state, then the enzyme can be subsequently activated, for instance by the addition of magnesium ion. The fragmented nucleic acids embedded and stored in the subject fiber preserve the genetic information, and specifically the sequence order of individual fragments. Such fibers have a variety of utility in, e.g., forensics and parental diagnosis.

The isolated fibers and arrays of fibers provide an effective tool for storing biopolymers including nucleic acids, proteins, lipids, carbohydrates and combinations thereof. The fiber arrays are particularly useful for storing genetic information from a variety of sources. Representative fiber arrays include organism array, mammalian array, human array, tissue array, and chromosome array.

The “organism array” of the subject invention comprises multiple unique fibers embedded therein biomolecules representative of distinct biological organisms. Exemplary organisms include members of the plant or animal kingdom, and microorganisms such as viruses, bacteria, protozoa, and yeast.

The “mammalian array” contains a plurality of unique fibers embedded therein biomolecules representative of distinct mammals. Non-limiting examples of mammals are primates (e.g. chimpanzees and humans), cetaceans (e.g. whales and dolphins), chiropterans (e.g. bats), perrisodactyls (e.g., horses and rhinoceroses), rodents (e.g. rats), and certain kinds of insectivores such as shrews, moles and hedgehogs. One variation of this specific type of array is a “human array”, in which the majority of the fibers of this array contain biomolecules of human origin.

The “tissue array” embodied in the present invention comprises a plurality of fibers embedded therein biomolecules predominantly present in specific body tissues. The types of body tissues include but are not limited to blood, muscle, nerve, brain, heart, lung, liver, pancreas, spleen, thymus, esophagus, stomach, intestine, kidney, testis, ovary, hair, skin, bone, breast, uterus, bladder, spinal cord and various kinds of body fluids. Non-limiting exemplary body fluids include urine, blood, spinal fluid, sinovial fluid, ammoniac fluid, cerebrospinal fluid (CSF), semen, and saliva.

Another type of fiber array (“chromosome array”) comprises fibers containing distinct nucleic acids from different chromosomes. The fiber may contain a portion or the entire distinct chromosome.

Another type of fiber array embodied in the present invention is a “personal fiber array”, which comprises unique biomolecules derived from individuals of a family, or individuals from different generations within the same pedigree. Such biomolecules can be DNA (e.g., chromosomal DNA, genomic DNA, cDNA, or a fragment thereof), RNA or a combination thereof that are derived from an individual. Preferably, the personal fiber array stores genetic information unique to a given individual. Fiber arrays of this category are especially useful for forensic and parental identification.

Yet another type of invention fiber array is one that comprises biomolecules such as nucleic acids associated with a particular disease or with a specific disease stage (i.e., “disease array”).

The present invention also provides a method of analyzing the biomolecule isolated by the subject method. The process typically involves providing a fiber embedded therein an isolated biomolecule that is configured to produce a detectable signal; and detecting the signal.

A variety of methods are available in the art for detecting embedded biomolecules, and signals generated therefrom. For examples, detection can be effected by the application of electromagnetic radiation (including light, X-rays, and Gamma Rays), electrons, neutrons, or other particles including ion beams. In some cases, electromagnetic radiation is used to stimulate the biomolecule or associated label and followed by detecting the emitted signals from the biomolecule. For instance, x-ray photoelectron spectroscopy (XPS) involves stimulating a sample with x-rays and measuring the energy of the electrons that are ejected. Conversely, x-ray fluorescence (XRF) is light stimulated by the stimulation of the molecule with electrons.

The detection methods will typically depend on the choice of labels used. As disclosed above, detectable labels suitable for use in the present invention include any composition detectable by spectroscopic, photochemical, biochemical, immunochemical, electrical, optical or chemical means. A wide variety of appropriate detectable labels are known in the art, which include but are not limited to luminescent labels, fluorescent labels, radioactive isotope labels, enzymatic or other ligands.

In some embodiments, the signal is generated by the interaction between the biopolymer and the binding agent from fluorescence resonance energy transfer (FRET) between fluorophores.). In FRET, an excited fluorophore (the donor) transfers its excited state energy to a light absorbing molecule (the acceptor) in a distance-dependent manner. The limitation on the distance over which the energy can travel allows one to discern the interactions between labeled molecules and entities in close proximity. Either the unit or the proximate compound/agent may be labeled with either the donor or acceptor fluorophore. FRET is the transfer of photonic energy between fluorophores. FRET has promise as a tool in characterizing molecular detail because of its ability to measure distances between two points separated on the order of 0.1 nanometer to 10 nanometers. The resolving power of FRET arises because energy transfer between donor and acceptor fluorophores is dependent on the inverse sixth power of the distance between the probes.

In general, optimal efficient FRET signal involves an efficient donor emission in the absence of acceptors and an efficient generation of a change in either donor or acceptor emissions during FRET. In some embodiments, a fluorophore is attached to the isolated biopolymer, and another fluorophore is attached to a binding agent which binds to a specific portion of the biopolymer. Thus, the presence of the FRET signal indicates that binding has occurred, and that the biomolecule contains the specific portion of interest. These embodiments can be used to determine the structure and sequence of the isolated biomolecule.

Representative donors and acceptors capable of fluorescence energy transfer include, but are not limited to, 4-acetamido-4′-isothiocyanatostilbene-2,2′disulfonic acid; acridine and derivatives: acridine, acridine isothiocyanate; 5-(2′-aminoethyl)aminonap-hthalene-1-sulfonic acid (EDANS); 4-amino-N-[3-vinylsulfonyl)phenyl]naphth-alimide-3,5 disulfonate; N-(4-anilino-1-naphthyl)maleimide; anthranilamide; BODIPY; Brilliant Yellow; coumarin and derivatives: coumarin, 7-amino-4-methylcoumarin (AMC, Coumarin 120), 7-amino-4-trifluoromethylcouluarin (Coumaran 151); cyanine dyes; cyanosine; 4′,6-diaminidino-2-phenylindole (DAPI); 5′,5″-dibromopyrogallol-sulfonaphthalein (Bromopyrogallol Red); 7-diethylamino-3-(4′-isothiocyanatophenyl)4-methylcoumarin; diethylenetriamine pentaacetate; 4,4′-diisothiocyanatodihydro-stilbene-2,-2′-disulfonic acid; 4,4′-diisothiocyanatostilbene-2,2′-disulfonic acid; 5-[dimethylamino]naphthalene-1-sulfonyl chloride (DNS, dansylchloride); 4-dimethylaminophenylazophenyl-4′-isothiocyanate (DABITC); eosin and derivatives: eosin, eosin isothiocyanate, erythrosin and derivatives: erythrosin B, erythrosin, isothiocyanate; ethidium; fluorescein and derivatives: 5-carboxyfluorescein (FAM), 5-(4,6-dichlorotriazin-2-yl)amin-ofluorescein (DTAF), 2′,7′-dimethoxy4′5′-dichloro-6-carboxyfluorescein (JOE), fluorescein, fluorescein isothiocyanate, QFITC, (XRITC); fluorescamine; IR144; IR1446; Malachite Green isothiocyanate; 4-methylumbelliferoneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron.TM. Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′-tetramethyl-6-carboxyrhodamine (TAMRA); tetramethyl rhodamine; tetramethyl rhodamine isothiocyanate (TRITC); riboflavin; rosolic acid; terbium chelate derivatives; Cy 3; Cy 5; Cy 5.5; Cy 7; IRD 700; IRD 800; La Jolla Blue; phthalo cyanine; and naphthalo cyanine.

The subject devices, including various forms of fiber arrays and the associated optical systems, are particularly suited for multiplexed single-molecule sequencing. Accordingly, the present invention provides methods of sequencing a target nucleic acid. In one embodiment, the method involves (a) elongating a biopolymer in a flowing medium; (b) subjecting the target nucleic acid molecule to an endonuclease or exonuclease reaction to yield sequence of cleaved fragments; (c) fixing the fragments in a fiber; and (c) detecting the cleaved fragments. In some embodiments, a 3′ to 5′ exonuclease is used resulting in single nucleotides as the cleaved units, which detected either within or outside the fiber if the cleaved units are released to the outside of the fiber. The subject sequencing methods can be used to determine the sequence of any nucleic acid molecules, including double-stranded or single-stranded, linear or circular nucleic acids (e.g., circular DNA), single stranded DNA hairpins, DNA/RNA hybrids, RNA with a recognition site for binding of the polymerase, or RNA hairpins. The methods of the present invention are suitable for sequencing complex nucleic acid structures, such as 5′ or 3′ non-translation sequences, tandem repeats, exons or introns, chromosomal segments, whole chromosomes or genomes.

In one aspect, the temporal order of base cleavage during the exonuclease reaction is registered on a single molecule of nucleic acid. Such registering step records the individual nucleotide units in the target nucleic acid that has been cleaved. The registering step may take place while the cleavage is in progression or subsequent to such event so long as a time sequence of the order of the unit being cleaved can be constructed. The individual nucleotide building blocks can be labeled prior to the exonuclease reaction. Where desired, the four different types of building block (namely, A, T, C, G) can be labeled with a distinct label.

Exonucleases are enzymes that cleave nucleotides one at a time from an end of a polynucleotide chain. These enzymes hydrolyze phosphodiester bonds from either the 3′ or 5′ terminus of polynucleotide molecules. A wide variety of exonucleases can be utilized in practicing the subject method. Non-limiting examples include exonuclease 1, and T4 exonuclease. The sequencing procedures of the present invention are performed under any conditions such that the order of the nucleotide units can be constructed. In one aspect, the reactants used for the relevant reaction (e.g., exonuclease reaction) are provided and adjusted to a physiologically relevant concentration. The conditions useful for performing a nuclease reaction are known in the art.

In some embodiments, an exonuclease capable of cleaving the individual units in the target nucleic acid is anchored within the field of detection. The size of the field of detection will depend on the choice of the detection means. Where optical confinements such as waveguides are used, the exonuclease is preferably placed under the effective observation volume of a given optical confinement.

Endonucleases cleave DNA in the middle of a segment rather than from the end. Restriction endonucleases cut only double-helical segments that contain a particular nucleotide sequence, and they make the incisions only within that sequence known as a recognition sequence. A wide variety of restriction endonucleases can be used including Type I, Type II, Type III and Type IV. Non-limiting examples of restriction endonucleases include EcoRI, BamHI, HindIII, MstII, TaqI, NotI, HinfI, Sau3A, PovII, SmaI, HaeIII, and AluI.

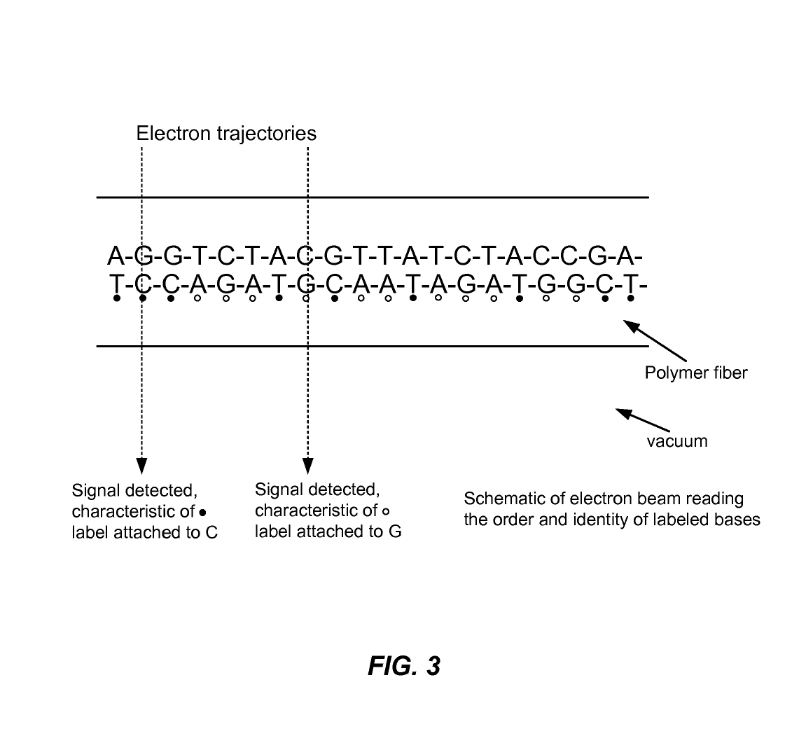

One aspect of the invention is the sequencing of biopolymers by elongating the biopolymers such that the sequence of individual units of the biopolymer can be determined. For example, where the biopolymer is a nucleic acid, the nucleic acid can be aligned and oriented along the length of the fiber such that a significant portion of the polymer chain is oriented along the axis of the fiber. (see Bellan et al. Nano Lett. 6(11), 2006). Electron microscopy has the capability of atomic scale resolution. In one aspect of the invention, nucleotides within an isolated, elongated, oriented nucleic acid are labeled with a label that is detectable in an electron microscope. In some embodiments, only one of each of the 4 bases is labeled, allowing the position of that base to be established. In other embodiments 2, 3, or all 4 of the bases is labeled with labels that can be distinguished from one another, for instance, where each of the bases is labeled with a different heavy element. In some embodiments, the identity of the specific heavy elements can be determined, for example by electron energy loss spectroscopy (EELS). FIG. 3 shows a schematic for measuring the sequence in an aligned, elongated, oriented nucleic acid. A similar method can be used to determine the sequence or structure of isolated polypeptides, for instance by associating specific amino acids with labels observable in the electron microscope.

As noted above, the subject fibers and associated systems are also useful for conducting and/or monitoring chemical reactions involving molecule-molecule interactions. Accordingly, the present invention provides a method of detecting the presence of an interaction involving a target biopolymer and a probe. The method typically involves the steps of providing a fiber embedded therein an isolated biopolymer; contacting the probe with the biopolymer under conditions sufficient to produce a stable probe-target biopolymer complex; and detecting the formation of the stable probe-target complex. In one aspect, the interaction is between a nucleotide polymer and a nucleic acid probe. Such interaction typically involves hybridization that yields a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. Florescent In-Situ Hybridization (FISH) can be conducted with the subject fibers to detect morphology of chromosome, and relate microscopic topological information to gene activity at the DNA, mRNA and protein level. FISH involves treating a chromosome with a relatively large (kilobase to megabase) fluorescently labeled nucleic acid probe. The probe binds by hybridization specifically to certain regions of the chromosome.

In some embodiments the binding agent will bind by hybridization. For example, where the biopolymer is a nucleic acid, a binding agent which can hybridize to the nucleic acid can be added. The hybridizing binding agent may have a label and may be added before, during or after fiber formation. In some embodiments the binding agent will hybridize with a complementary section of the biopolymer. The term “complementary” refers to the topological compatibility or matching together of interacting surfaces of a binding agent and biopolymer. Complementary includes base complementary such as G is complementary to C and A is the complement of T or U in the genetic code. Complementary also includes other forms of ligand-receptor (also known as ligand-anti-ligand) interactions, such as between other types of receptors and their agonists, antagonists, and other molecules that bind thereto or show some affinity for. Depending upon the stringency of the hybridization conditions used, the binding agent may hybridize to sequences more closely or more distantly related to the biopolymer. Thus, the sequence on the biopolymer can be one that hybridizes under a selected set of hybridization conditions to a binding agent having the reference sequence.

Hybridization conditions can be varied in order to utilize different lengths of hybridization and to tolerate a certain amount of mismatch. In some cases, the binding agents will hybridize to only 2, 3 4, 5, 6, 8, 10, 20, 40, or 60 base pairs, and in other cases the binding agents will contain hundreds to thousands of bases.

In another embodiment of the invention, hybridization of binding agents containing labels visible by electron microscopy are used to label isolated elongated nucleic acid biopolymers in order to determine structure and sequence of the nucleic acid. Electron microscopy, particularly TEM, has the capability of resolution on the order of single atoms. In one embodiment, nucleic acids are highly elongated such that portions of the molecule lie linearly along the fiber axis, and the nucleic acids are associated with binding agents containing labels visible by electron microscopy such that the sequence and/or structure of the nucleic acid can be determined. In these embodiments utilizing TEM, the binding agents can associate with less than about 2, 3, 4, 5, 6, 8, 10, 20, 40, or 60 base pairs on the isolated nucleic acid biopolymer.

Suitable hybridization conditions for the practice of the present invention are such that the recognition interaction between the probe and target is both sufficiently specific and sufficiently stable. Hybridization reactions can be performed under conditions of different “stringency”. Relevant conditions include temperature, ionic strength, time of incubation, and the washing procedure. Higher stringency conditions are those conditions, such as higher temperature and lower sodium ion concentration, which require higher minimum complementarity between hybridizing elements for a stable hybridization complex to form. Conditions that increase the stringency of a hybridization reaction are widely known and published in the art. See, for example, (Nonradioactive In Situ Hybridization Application Manual, Boehringer Mannheim, second edition).

In general, there is a tradeoff between hybridization specificity (stringency) and signal intensity. In a preferred embodiment, washing the hybridized array prior to detecting the target-probe complexes is performed to enhance the noise-signal ratio. Typically, the hybridized array is washed at successively higher stringency solutions and signals are read between each wash. Analysis of the data sets thus produced will reveal a wash stringency above which the hybridization pattern is not appreciably altered and which provides adequate signal for the particular polynucleotide probes of interest. Parameters governing the wash stringency are generally the same as those of hybridization stringency. Other measures such as inclusion of blocking reagents (e.g. sperm DNA, detergent or other organic or inorganic substances) during hybridization can also reduce non-specific binding.

For a convenient detection of the probe-target complexes formed during the hybridization assay, the nucleotide probes are conjugated to a detectable label. Detectable labels suitable for use in the present invention include any composition detectable by spectroscopic, photochemical, biochemical, immunochemical, electrical, optical or chemical means. A wide variety of appropriate detectable labels are known in the art, which include luminescent labels, radioactive isotope labels, enzymatic or other ligands.

In another aspect, the subject fibers and the associated systems can be used to detect an interaction between a nucleotide polymer and a proteinaceous probe. This is particularly useful for screening candidate proteinaceous probes such as transcription factors, translation initiation factors, and suppressors that are capable of binding to defined regulatory or coding sequences of a target nucleic acid.

In another aspect, the subject fibers and the associated systems can be used to detect an interaction between a target polypeptide and a nucleic acid sequence, or between a target polypeptide and a proteinaceous probe.

In some embodiment, the reaction is performed by contacting the proteinaceous probe with a fiber array of particular interest under conditions that will allow a complex to form between the probe and the target. The formation of the complex can be detected directly or indirectly according standard procedures in the art. In the direct detection method, the probes are supplied with a detectable label and unreacted probes may be removed from the complex; the amount of remaining label thereby indicating the amount of complex formed. For such method, it is preferable to select labels that remain attached to the probes even during stringent washing conditions. It is more desirable, however, that the label does not interfere with the binding reaction. In the alternative, an indirect detection procedure requires the probe to contain a label introduced either chemically or enzymatically, that can be detected by affinity cytochemistry. A desirable label generally does not interfere with target binding or the stability of the resulting target-probe complex. A wide variety of labels are known in the art. Non-limiting examples of the types of labels which can be used in the present invention include radioisotopes, enzymes, colloidal metals, fluorescent compounds, bioluminescent compounds, chemiluminescent compounds and any other labels disclosed herein.

The amount of probe-target complexes formed during the binding reaction can be quantified by a variety of quantitative assays and use detection systems disclosed herein or available in the art. As illustrated above, the formation of probe-target complex can be measured directly by the amount of label remained at the site of binding. In an alternative, the target protein is tested for its ability to compete with a labeled analog for binding sites on the specific probe. In this competitive assay, the amount of label captured is inversely proportional to the amount of target.

In some embodiments, the fiber can be cut in order to remove sections of the fiber for further analysis or storage. This cutting of the fiber is analogous to the way that analysis gels such as electrophoresis gels, are often cut in order to isolate a portion of the molecular population within them. The biomolecule or biomolecules within the cut portion of the gel can then be subjected to further analysis. For example, where the biomolecule is a nucleic acid, the biomolecule can be amplified, for example by polymerase chain reaction (PCR) and analyzed, for example by sequencing.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### EXAMPLES

### Example 1

### Preparation of Electrospinning Solution

Approximately 15 μL of stock bacteriophage DNA (New England BioLabs) solution was added to a solution of 1500 μL of buffer (10 mM HEPES, 10 mM NaCl) and 1.5 μL of stock YOYO-1 fluorescent dye solution (Molecular Probes), yielding a nominal labeling ratio of about 7.6:1. This solution was incubated at about 65° C. for about 15-20 minutes to linearize the DNA and then filtered through BioSpin 6 spin columns (Bio-Rad) to remove unincorporated dye. Then approximately 100 μL of this solution was added to 900 μL of buffer (10 mM HEPES, 10 mM NaCl), 20 mg poly-L-aspartic acid (Sigma P6762) and 20-100 mg DABCO (Sigma). The poly-L-aspartic acid was added to prevent the DNA from collapsing into bundles. The DABCO was added to reduce photobleaching. This solution was gently mixed for several minutes. Finally, approximately 200 mg of polyethylene oxide (PEO) (MW 100,000, Sigma) was added and the solution was gently mixed overnight to allow the polymer to fully dissolve. The above procedure yielded a DNA concentration of roughly 3.7×10¹⁰ molecules per cm³ of solid, corresponding to a linear density of approximately 1 molecule/mm for 200 nm diameter fibers. Control solutions were also made that were identical to the above solution except that they lacked a) labeled DNA or b) poly-L-aspartic acid.

### Example 2

### Electrospinning of Nanofibers

Nanofibers from the solutions described in Example 1 were deposited using the scanned electrospinning method. The electrospinning tip was a microfabricated silicon chip coated with a thin gold layer. Drops of the solution were manually placed on the electrospinning tip before turning on the electrospinning voltage source. We used a voltage of 7-10 kV over a distance of about 4 cm to form the electrospinning jet. The collecting substrate was attached to a motor so that the nanofibers would be oriented and isolated. The resulting fiber diameters varied from approximately 100-350 nm as measured by atomic force microscopy (AFM).

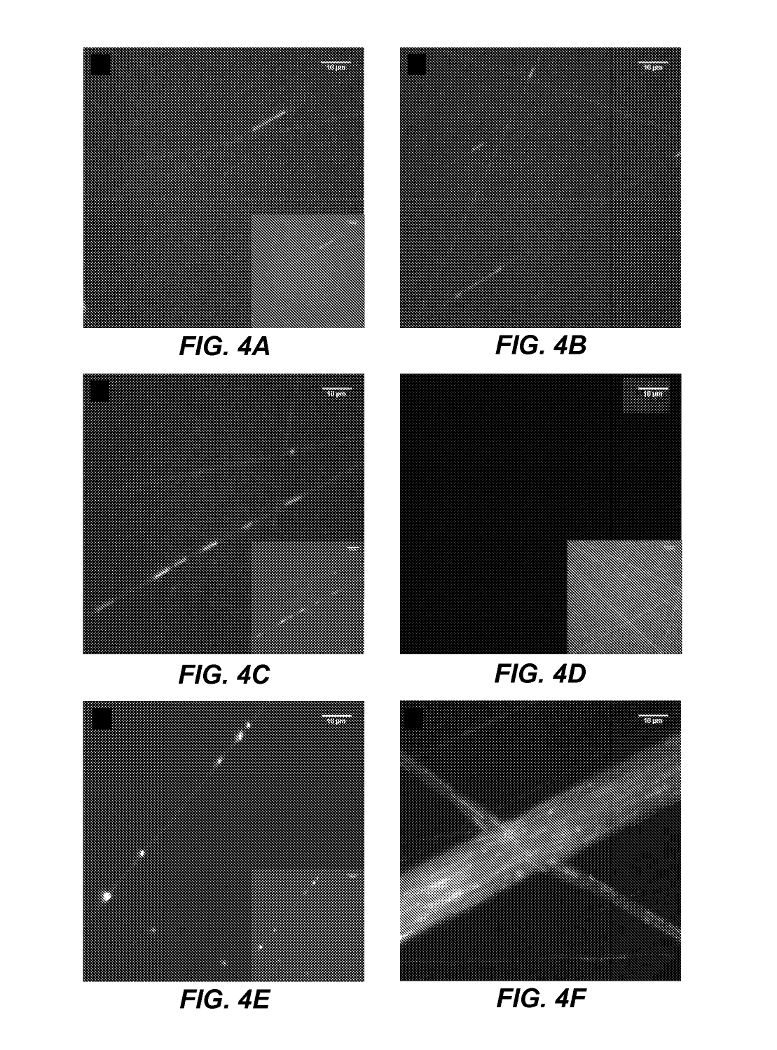

The water-soluble fibers were spun onto glass coverslips so that they could be imaged from below with a 60×1.20 NA water-immersion objective. The resulting fibers were imaged using an inverted fluorescence microscope (Olympus IX70, EXFO X-cite 120 illuminator, Omega XF100-2 filter cube) using a Cascade 512b EMCCD camera (Roper Scientific). Examples of the resulting images are shown in (FIGS. 4a-c). Fibers were also electrospun from two control solutions that were identical to the sample solution except that they lacked a) labeled DNA (FIG. 4d) or b) poly-L-aspartic acid (FIG. 4e). Because the fibers formed from the solution lacking DNA showed no isolated discrete fluorescent lines (other than uniform background fluorescence from the PEO fibers themselves), we conclude that the fluorescent lines represent labeled DNA molecules. The fibers formed from the solution lacking poly-L-aspartic acid contained highly fluorescent blobs (large DNA bundles), but showed few fluorescent lines, indicating that the poly-L-aspartic acid assisted in the elongation of the DNA. We also prepared fibers containing DNA that was not fluorescently labeled and, as expected, observed no fluorescence above that of the PEO fiber autofluorescence. Occasionally the electrospinning jet became particularly unstable, ejecting and depositing a large ribbon of material on the glass substrate. These large ribbons contain several stretched aligned DNA molecules (FIG. 4f).

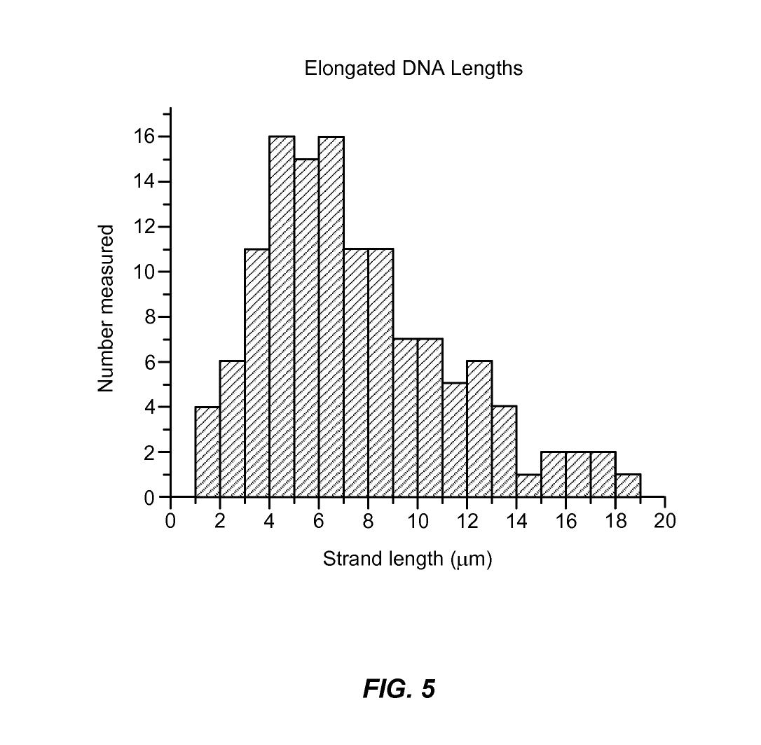

The lengths of the DNA strands were measured manually using the image processing software ImageJ (Rasband, W. S., ImageJ, U. S. NIH, Bethesda, Md., USA, http://rsb.info.nih.gov/ij/, 1997-2006). A histogram of the resulting data for 54 images (129 molecules) from one coverslip is shown in FIG. 5. The lengths were measured from one end of a molecule to the other, including any small dark regions between the ends in which the molecule may have fragmented. Molecules that exhibited dark regions that were of the same size or larger than the fluorescent regions were not counted. It is possible that some of the shorter measured lengths were actually fragments of a larger molecule, or that some of the longer lengths counted as individual molecules were actually DNA concatomers. Brighter spots on the molecule may be due to bunching or folding. The full contour length of λ DNA (nominally 16.3 μm) labeled with YOYO-1 at a base pair to dye labeling ratio of approximately 4:1 has been reported as 22 μm. Given that each YOYO-1 dye molecule extends the chain by 0.4 nm, we expect a contour length of 18.8 μm for the labeling ratio we used.

### Example 3

### Elongated DNA Fluid Dynamic Behavior in the Fiber

To better understand the fluid dynamic behavior of the DNA in the PEO solution while in the electrospinning jet under these particular conditions, we also measured the relaxation time of the labeled DNA molecules in the bulk PEO solution. The viscous electrospinning solution was introduced into a fused-silica microchannel device (50 μm wide and 750 nm deep) and the DNA was driven with an electric field (100-200 V/cm), causing it to experience a sheer force and elongate. The field was then turned off and videos were recorded of the elongated DNA relaxing into a blob. Previous studies have used similar methods to study the relaxation behavior of DNA in a viscous solution (See Smith et al., Science, 281, 1335, 1998) and calculated relaxation times of 4-17 seconds (depending on the solution viscosity) using an exponential decay model. Our videos were processed with homemade routines in Matlab (The Mathworks) and the DNA length vs. time data was fit to a decaying exponential using Origin 7.5 (OriginLab), yielding a time constant ranging from 2.1 to 19 sec (mean 8±5 sec) over 20 samples, depending on whether the DNA was sticking to a surface. If we then calculate the Deborah number using previously published order-of-magnitude estimates of the overall strain rate in a whipping electrospinning jet, 10⁵ sec−1, the resulting value of De≈105-106 suggests that we should expect to see the DNA molecules elongate in the jet. Even in the straight section of the jet, the order-of-magnitude estimated strain rate of 10 sec−1 yields De≈10-100. Under these conditions, the overall strain rates in the electrospinning jet can be on or above the order of magnitude necessary for DNA chain scission in an elongational flow. (see Atkins, et al., Biopolymers, 32(8), 911, 1992). By varying the conditions used to create the electrospun fiber including variation of the voltage, and the viscosity of the pre-fiber fluid, the resulting strain rate can be varied such that the molecules are not elongated, such that they are slightly elongated, such that they are substantially fully elongated, and even to the extent where fragmentation of the molecules occurs.

1.-24. (canceled)

25. A method for obtaining nucleic acid sequence information, comprising:
(a) scanning with an electron beam a population of labeled nucleic acid molecules on a substrate, wherein individual nucleic acid molecules of said population of labeled nucleic acid molecules are fixed within a fiber material that is substantially transparent to said electron beam, and wherein said population of labeled nucleic acid molecules is deposited from a tip onto said substrate that is moving in a direction such that said labeled nucleic acid molecules within said population are elongated on said substrate and aligned along said direction of movement of said tip;
(b) detecting bases of said individual nucleic acid molecules of said population of labeled nucleic acid molecules while scanning said population of labeled nucleic acid molecules with said electron beam; and
(c) generating sequence information of said individual nucleic acid molecules detected in (b), wherein said sequence information includes an identity and order of bases within each of said individual nucleic acid molecules.

26. The method of claim 25, wherein nucleic acid molecules of said population are labeled prior to depositing said nucleic acid molecules of said population on said substrate.

27. The method of claim 25, wherein nucleic acid molecules of said population are labeled after depositing said nucleic acid molecules of said population on said substrate.

28. The method of claim 25, wherein said population of labeled nucleic acid molecules is deposited on said substrate using an electrical potential on said tip relative to said substrate.

29. The method of claim 25, wherein nucleic acid molecules of said population comprise deoxyribonucleic acid.

30. The method of claim 25, wherein two, three or four types of bases of nucleic acid molecules of said population are labeled.

31. The method of claim 25, wherein nucleic acid molecules of said population are labeled with one or more heavy metal atoms.

32. The method of claim 31, wherein said one or more heavy metal atoms comprise gold, iron, lead, iridium, cobalt, mercury, osmium, silver, mercury, uranium, platinum, rhodium, ruthenium, palladium, copper, or selenium.

33. The method of claim 25, wherein said population of labeled nucleic acid molecules is detected using electron energy loss spectroscopy to determine an identity of labels of said population of labeled nucleic acid molecules.

34. The method of claim 25, wherein said fiber material comprises a polymer.

35. The method of claim 34, wherein said polymer is a water soluble polymer.

36. A method for obtaining nucleic acid sequence information, comprising:
(a) providing a substrate having a nucleic acid molecule deposited on a surface thereof, wherein said nucleic acid molecule is elongated on said surface, wherein said nucleic acid molecule is fixed within a fiber material that is substantially transparent to an electron beam, and wherein one or more types of bases of said nucleic acid molecule include a detectable label;
(b) scanning said nucleic acid molecule with said electron beam;
(c) while scanning said nucleic acid molecule with said electron beam, resolving signals from said surface via electron energy loss spectroscopy (EELS), wherein said signals are indicative of an identity and order of said bases within said nucleic acid molecule; and
(d) using said signals resolved in (c) to obtain sequence information of said nucleic acid molecule, wherein said sequence information includes said identity and order of said bases within said nucleic acid molecule.

37. The method of claim 36, further comprising depositing and elongating said nucleic acid molecule on said surface prior to (a).

38. The method of claim 36, further comprising scanning a plurality of elongated nucleic acid molecules deposited on said substrate surface.

39. The method of claim 36, wherein said detectable label comprises one or more heavy metal atoms.

40. The method of claim 39, wherein said one or more heavy atoms comprise gold, iron, lead, iridium, cobalt, mercury, osmium, silver; mercury, uranium, platinum, rhodium, ruthenium, palladium, copper, or selenium.

41. The method of claim 36, wherein said nucleic acid molecule comprises deoxyribonucleic acid.

42. The method of claim 36, wherein two, three or four types of bases of said nucleic acid molecule include detectable labels.

43. The method of claim 36, wherein said fiber material comprises a polymer.

44. The method of claim 43, wherein said polymer is a water soluble polymer.
